SEQUENCE LISTING (14 sequences)

SEQ ID NO: 1 - DNA, 20 bp, Artificial Sequence
  Artificial oligonucleotide primer
ggcgaaacatgacgaaaaac

SEQ ID NO: 2 - DNA, 25 bp, Artificial Sequence
  Artificial oligonucleotide primer
cctcgttatgtagcaatcgtattac

SEQ ID NO: 3 - DNA, 17 bp, Artificial Sequence
  Artificial oligonucleotide primer
ttacgtgacgtcccatc

SEQ ID NO: 4 - DNA, 17 bp, Artificial Sequence
  Artificial oligonucleotide primer
tgcgacatgggtacaac

SEQ ID NO: 5 - DNA, 23 bp, Artificial Sequence
  misc_feature 1: FAM
ncaaccatcgtcatcgtcaattn

SEQ ID NO: 6 - DNA, 28 bp, Artificial Sequence
  Artificial oligonucleotide primer
aaaaggtaacaaattacttagttgatgg

SEQ ID NO: 7 - DNA, 27 bp, Artificial Sequence
  Artificial oligonucleotide primer
cgaagttaaattactcccttcttcctt

SEQ ID NO: 8 - DNA, 20 bp, Artificial Sequence
  Artificial oligonucleotide primer
gggttatatgttccagaatc

SEQ ID NO: 9 - DNA, 22 bp, Artificial Sequence
  Artificial oligonucleotide primer
gtaactaaatcagattggttct

SEQ ID NO: 10 - DNA, 28 bp, Artificial Sequence
  misc_feature 1: FAM
ngaccttcaaaaggtgtagaattaaggn

SEQ ID NO: 11 - DNA, 25 bp, Artificial Sequence
  Artificial oligonucleotide primer
aatatcaataaccttacagcaaccc

SEQ ID NO: 12 - DNA, 23 bp, Artificial Sequence
  Artificial oligonucleotide primer
atgcattaacctaaaggcttctg

SEQ ID NO: 13 - DNA, 17 bp, Artificial Sequence
  Artificial oligonucleotide probe
ccatcgtcatcgtcaat

SEQ ID NO: 14 - DNA, 21 bp, Artificial Sequence
  Artificial oligonucleotide probe
tcaaaaggtgtagaattaagg

(19) United States
(12) Patent Application Publication
(10) Pub. No.: US 2008/0176220 A1
(43) Pub. Date: Jul. 24, 2008

(54) Method, Kit and System for Enhanced Nested Pcr

(75) Inventors: Gert Bolander Jensen, Copenhagen (DK); Lars Thomsen, Alborg (DK); Oene Robert Veltman, Alborg (DK)

Correspondence Address: KNOBBE MARTENS OLSON & BEAR LLP, 2040 MAIN STREET, FOURTEENTH FLOOR, IRVINE, CA, 92614, US

(73) Assignee: THOMSEN BIOSCIENCE, NORRESUNBY (DK)

(21) Appl. No.: 10/590,648

(22) PCT Filed: Feb. 25, 2005
(86) PCT No.: PCT/DK05/00131

§ 371 Date: Nov. 16, 2007

(30) Foreign Application Priority Data
Feb. 26, 2004 (DK) 2004-00304

(51) Int. Cl. C12Q 1/68; C12M 1/34

(52) U.S. Cl. 435/6; 435/287.2

(57) ABSTRACT

The present invention relates to a method, a kit and a system for sensitive detection of a target nucleic acid molecule. The target nucleic acid molecule may e.g. be specific for a certain microorganism, e.g. a pathogenic microorganism such as B. anthracis. The methods and kits relate to so-called nested PCR and especially improvements, which renders the nested PCR technique better suited for automated analysis.

### FIELD OF THE INVENTION

The present invention relates to a method of and a kit for sensitive detection of a target nucleic acid molecule. The target nucleic acid molecule may e.g. be specific for a certain microorganism, e.g. a pathogenic microorganism such as B. anthracis. The methods and kits relate to so-called nested PCR and especially improvements, which renders the nested PCR technique better suited for automated analysis.

### BACKGROUND

Rapid and sensitive detection of a target nucleic acid molecule has many important applications and plays e.g. an important role in the detection of vira and pathogenic microorganisms such as Bacillus anthracis.

Bacillus anthracis, the causative agent of Anthrax, is a large, aerobic, Gram-positive, spore-forming, non-motile Bacillus. Spores are formed in culture, in the soil, and in the tissues and exudates of dead animals when there is limited access to nutrients, and subsequently not in the blood or tissues of living animals. Spores can remain viable in soil for decades. The bacterium ordinarily produces a zoonotic disease in domesticated and wild animals such as goats, sheep, cattle, horses, and swine. Humans become infected by the cutaneous route (direct contact with diseased animals, industrial work with hides, wool, brushes, or bone meal), by inhalation (Woolsorter's disease), or by ingestion (meat from diseased animals).

Anthrax endospores do not divide, have no measurable metabolism, and are very resistant to drying out, heat, ultraviolet light, gamma radiation, and many disinfectants. All known anthrax virulence genes are expressed by the vegetative form of B. anthracis upon germination of spores within the body of the host. Endospores introduced into the body by abrasion, inhalation, or ingestion are phagocytized by macrophages and carried to regional lymph nodes. The endospores germinate inside the macrophages and become vegetative bacteria; the vegetative bacteria are then released from the macrophages, multiply in the lymphatic system, and enter the bloodstream until there is as many as 10⁷ to 10⁸ organisms/ml blood, causing massive septicemia. Once they have been released from the macrophages, there is no evidence that an immune response is initiated against vegetative bacilli. Anthrax bacilli express virulence factors, including toxin and capsule polypeptides. The resulting toxemia has systemic effects that lead to the death of the host.

Several approaches for sensitive detection of microorganisms such as B. anthracis by means of nested PCR have been disclosed in the prior art. Jackson et al. describes a study where tissue samples of Russian anthrax victims is analyzed for B. anthracis via PCR amplification. Jackson et al. discloses PCR amplification of the capA gene as well as nested PCR of the same.

Beyer et al. describes a nested PCR method for the detection of B. anthracis in environmental samples. The nested PCR method is designed to amplify a DNA sequence that comprises parts of both the capB and the capC gene. The PCR product is separated using gel electrophoresis and stained using ethidium bromide.

The concept of single-tube nested PCR is e.g. disclosed in Herrmann et al. Here single-tube nested PCR is used for Detection of Neisseria gonorrhoeae from air-Dried genital samples.

These approaches suffer from several disadvantages and require e.g. complex and time consuming handling of samples and reagents and are difficult to adapt to automated analysis systems such as biochips.

### SUMMARY OF THE INVENTION

An object of the present invention relates to the provision of methods, kits and systems for rapid and/or sensitive amplification or detection of a target nucleic acid molecule.

Another object of the present invention relates to the provision of energy saving methods, kits and systems for rapid and/or sensitive amplification or detection of a target nucleic acid molecule.

Yet another object of the present invention relates to the provision of simple and robust methods, kits and systems for rapid and/or sensitive amplification or detection of a target nucleic acid molecule. Preferably, such methods, kits and systems should require as few liquid handling steps as possible and should involve as few different reagents as possible.

Still another object of the present invention relates to the provision of methods, kits and systems for selective, rapid and sensitive detection of a target nucleic acid molecule related to Bacillus anthracis.

Other objects of the invention will become apparent when reading the description and the examples.

A broad aspect of the invention relates to a method of amplifying, and optionally also detecting, a target nucleic acid sequence (TNAS), the method comprising the steps of:
a) providing a sample that may or may not comprise a TNAS,
b) providing a pair of outer primers and a pair of inner primer, a nucleic acid polymerase and standard reagents for PCR, the melting temperature (Tm) of the pair of outer primers being at least 2° C. higher than the Tm of the pair of inner primers,
c) contacting the sample with the pair of outer primer and the pair of inner primers, and standard reagents for PCR, thus obtaining the reaction mixture,
d) cycling, at least two times, the temperature of the reaction mixture between a first denaturation temperature, a first annealing temperature and a first extension temperature, the first annealing temperature being similar to or lower than the lowest Tm of the outer primer pair and higher than the highest Tm of the inner primer pair,
e) cycling, at least two times, the temperature of the reaction mixture between a second denaturation temperature, a second annealing temperature and a second extension temperature, the second annealing temperature being similar to or lower than the lowest Tm of the inner primer pair, and
f) optionally, analyzing the product of step d and/or step e) to detect the presence of the TNAS.

Another aspect of the present invention related to a kit comprising a pair of outer primers and a pair of inner primer, the melting temperature (Tm) of the pair of outer primers being higher than the Tm of the pair of inner primers.

Yet another aspect of the invention relates to an analysis system for detection of a microorganism, the analysis system comprising a pair of outer primers and a pair of inner primer, the melting temperature (Tm) of the pair of outer primers being higher than the Tm of the pair of inner primers.

Yet further special aspects of the invention relate to methods and kits for amplifying and/or detecting a TNAS of B. anthracis.

### BRIEF DESCRIPTION OF THE FIGURES

In the following some embodiments of the present invention will be described with reference to the figures, wherein

FIG. 1 illustrates some examples of how the outer pair of primers and the inner pair of primers may bind to the nucleic acid sequence comprising the TNAS, and

FIG. 2 shows the effect of performing single-tube nested PCR relative to conventional PCR.

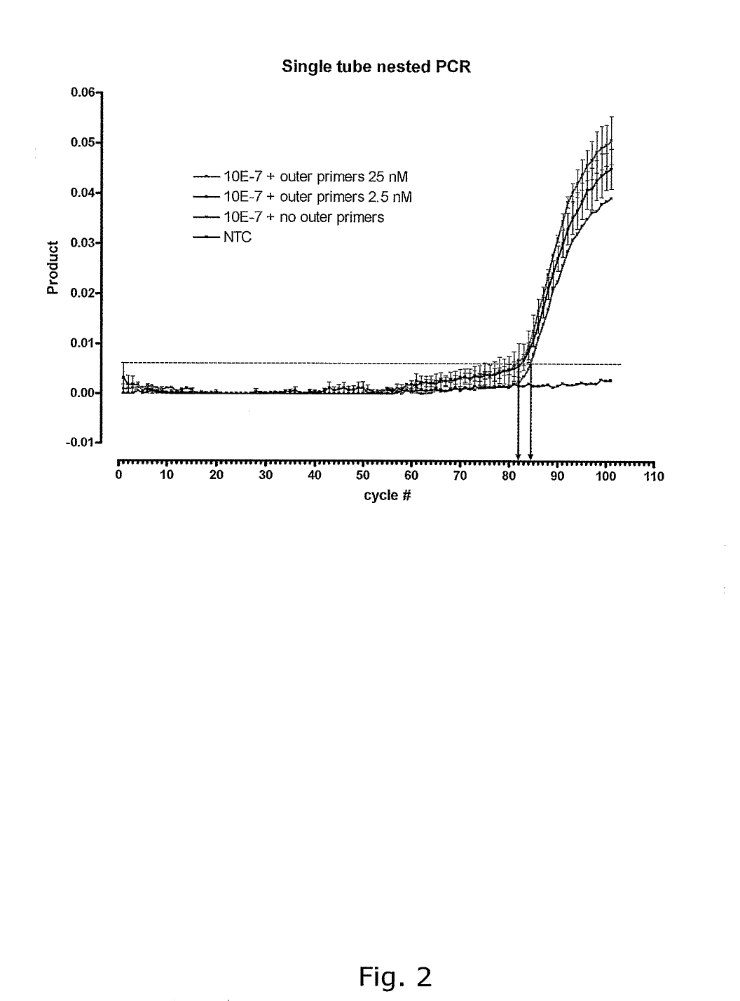

### DETAILED DESCRIPTION OF THE INVENTION

A broad aspect of the invention relates to a method of amplifying, and optionally also detecting, a target nucleic acid sequence (TNAS), the method comprising the steps of:
a) providing a sample that may or may not comprise a TNAS,
b) providing a pair of outer primers and a pair of inner primer, a nucleic acid polymerase and standard reagents for PCR, the melting temperature (Tm) of the pair of outer primers being at least 2° C. higher than the Tm of the pair of inner primers,
c) contacting the sample with the pair of outer primer and the pair of inner primers, and standard reagents for PCR, thus obtaining the reaction mixture,
d) cycling, at least two times, the temperature of the reaction mixture between a first denaturation temperature, a first annealing temperature and a first extension temperature, the first annealing temperature being similar to or lower than the lowest Tm of the outer primer pair and higher than the highest Tm of the inner primer pair,
e) cycling, at least two times, the temperature of the reaction mixture between a second denaturation temperature, a second annealing temperature and a second extension temperature, the second annealing temperature being similar to or lower than the lowest Tm of the inner primer pair,
f) optionally, analyzing the product of step d and/or step e) to detect the presence of the TNAS.

According to the present invention, the term “sample” relates to a substance, which may or may not comprise one or more compounds of interest. The sample may e.g. be a biological sample or a non-biological sample.

A biological sample may e.g. be selected from the group consisting of dermal swabs, cerebrospinal fluid, blood, sputum, bronchio-alveolar lavage, bronchial aspirates, lung tissue, and urine.

Non-biological samples may for example be powders, air samples, earth samples surface swipes, and rinse products from solid materials.

Biological or non-biological samples can be cultured. The culture then can be evaluated for the presence of e.g. a microorganism, such as B. anthracis, using the methods, kits, chips, devices and systems of the invention.

According to the present invention, the term “nucleic acid”, “nucleic acid sequence” and “nucleic acid molecule” should be interpreted broadly and may for example be an oligomer or polymer of ribonucleic acid (RNA) or deoxyribonucleic acid (DNA) or mimetics thereof. This term includes molecules composed of naturally-occurring nucleobases, sugars and covalent internucleoside (backbone) linkages as well as molecules having non-naturally occurring nucleobases, sugars and covalent internucleoside (backbone) linkages which function similarly or combinations thereof. Such modified or substituted nucleic acids may be preferred over native forms because of desirable properties such as, for example, enhanced cellular uptake, enhanced affinity for nucleic acid target molecule and increased stability in the presence of nucleases and other enzymes, and are in the present context described by the terms “nucleic acid analogues” or “nucleic acid mimics”. Preferred examples of nucleic acid mimetics are peptide nucleic acid (PNA-), Locked Nucleic Acid (LNA-), xylo-LNA-, phosphorothloate-, 2′-methoxy-, 2′-methoxyethoxy-, morpholino- and phosphoramidate-comprising molecules or functionally similar nucleic acid derivatives.

According to the present invention, the term “target nucleic acid sequence” (TNAS) relates to a nucleic acid sequence of special interest, e.g. for analytical or diagnostic purposes. The TNAS may be a gene or a fragment thereof or it may e.g. be an artificial nucleic acid sequence.

According to the present invention, the term “primer” relates to a nucleic acid sequence, which is capable of hybridizing e.g. to the TNAS, to a nucleic acid sequence in the vicinity of the target sequence, or to a nucleic acid sequence overlapping with the TNAS. Alternatively, a primer may be capable of hybridizing to either the complementary sequence of the TNAS, to the complementary sequence of a nucleic acid sequence in the vicinity of the TNAS, or to the complementary sequence of a nucleic acid sequence overlapping with the TNAS.

The primers typically comprise oligonucleotides, that is, nucleic acid molecules comprising in the range of 5-30 nucleotides, such as in the range of 5-10 nucleotides, 10-15 nucleotides, 15-20 nucleotides, 20-25 nucleotides, and 25-30 nucleotides. It is also envisioned that longer nucleic acid molecules may be used as primers. Thus, a primer may comprise a nucleic acid molecule comprising in the range of 30-50 nucleotides, such as in the range of 30-35 nucleotides, 35-40 nucleotides, 40-45 nucleotides, and 45-50 nucleotides.

For example, a primer may essentially consist of an oligonucleotide, that is, nucleic acid molecules essentially consisting of in the range of 5-30 nucleotides, such as in the range of 5-10 nucleotides, 10-15 nucleotides, 15-20 nucleotides, 20-25 nucleotides, and 25-30 nucleotides. Also, a primer may be a nucleic acid molecule essentially consisting of in the range of 30-50 nucleotides, such as in the range of 30-35 nucleotides, 35-40 nucleotides, 40-45 nucleotides, and 45-50 nucleotides.

The method of the present invention relates to the PCR technique nested PCR wherein at least two pairs of primers are used, that is, a pair of outer primers and a pair of inner primers. In a pair of primers, one primer, the sense primer, is typically designed to be capable of hybridizing to the nucleic acid strand comprising the TNAS strand. The other primer, the antisense primer, is typically designed to be capable of hybridizing to the complementary nucleic acid strand of the nucleic acid strand comprising the TNAS strand.

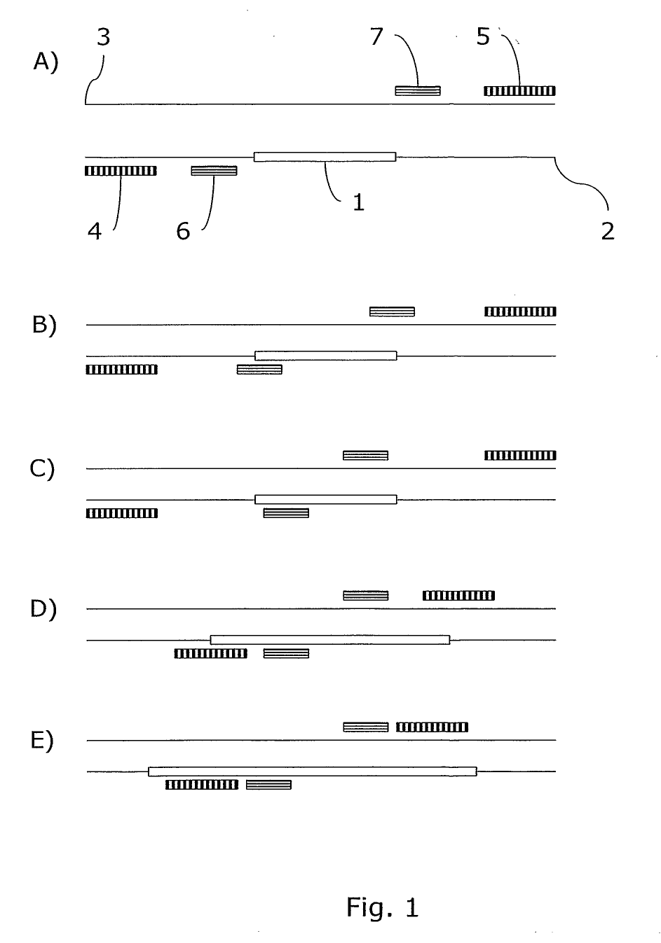

FIG. 1 shows some examples of how the pair of outer primers (4, 5) and the pair of inner primers (6, 7) may hybridize relative to the nucleic acid strand (2) comprising the TNAS (1) and relative to the nucleic acid strand (3) complementary to the nucleic acid strand comprising the TNAS. In FIG. 1 A) both the sense primers of outer pair of primers (4) and the inner pair of primers (6) hybridizes outside but in the vicinity of the TNAS (1) on the nucleic acid strand (2) comprising the TNAS. Also the antisense primers of outer pair of primers (5) and the inner pair of primers (7) hybridizes outside but in the vicinity of the TNAS (1) on the nucleic acid strand (2) comprising the TNAS.

In FIG. 1 B) the sense primer of the inner pair of primers overlaps with the TNAS when hybridized to the nucleic acid strand comprising the TNAS. The antisense primer of the inner pair of primers overlaps with the complementary sequence of the TNAS when hybridized to the complementary sequence of the nucleic acid strand comprising the TNAS.

In FIG. 1 C) the sense primer of the inner pair of primers hybridizes to a part of the TNAS when hybridized to the nucleic acid strand comprising the TNAS. The antisense primer of the inner pair of primers hybridizes with a part of the complementary sequence of the TNAS when hybridized to the complementary sequence of the nucleic acid strand comprising the TNAS.

In FIG. 1 D) the sense primer of the outer pair of primers overlaps with the TNAS when hybridized to the nucleic acid strand comprising the TNAS. The antisense primer of the outer pair of primers overlaps with the complementary sequence of the TNAS when hybridized to the complementary sequence of the nucleic acid strand comprising the TNAS.

In FIG. 1 E) the sense primer of the outer pair of primers hybridizes to a part of the TNAS when hybridized to the nucleic acid strand comprising the TNAS. The antisense primer of the outer pair of primers hybridizes with a part of the complementary sequence of the TNAS when hybridized to the complementary sequence of the nucleic acid strand comprising the TNAS.

According to the present invention, the term “nucleic acid amplification” relates to a process in which a template, e.g. a fragment of the nucleic acid comprising the TNAS, is copied into a number of copies.

Polymerase chain reaction (PCR) is one of the most commonly used nucleic acid amplification techniques. U.S. Pat. Nos. 4,683,202, 4,683,195, 4,800,159, and 4,965,188 disclose embodiments of the PCR technique. PCR typically employs two oligonucleotide primers that bind to a selected nucleic acid template (e.g., DNA or RNA). A primer can be purified from a restriction digest by conventional methods, or it can be produced synthetically. The primer is preferably single-stranded for maximum efficiency in amplification, but the primer can be double-stranded. Double-stranded primers are first denatured, i.e., treated to separate the strands. One method of denaturing double stranded nucleic acids is by heating.

Other methods of nucleic acid amplification include Strand Displacement Amplification (SDA), Ugation-Rolling Circle Amplification (L-RCA) and their combinations/modifications. These methods as well as PCR are well known to the person skilled in the art and are e.g. described in Sambrook et al.

The term “nucleic acid polymerase” or “thermostable polymerase” relates to a DNA- or RNA-dependent DNA polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from thermophilic or caldoactive strains such as Thermus flavus, T. ruber, T. thermophilus, T. aquaticus, T. lacteus, T. rubens, Thermococcus litoralis, Pyrococcus furiosus, Bacillus stearothermophilus and Methanothermus fervidus. Nonetheless, polymerases that are not thermostable also can be employed in PCR assays provided the enzyme is replenished.

If the DNA of the sample TNAS is hybridized to a complementary nucleic acid sequence, it is necessary to separate the two strands before the TNAS can be amplified in a PCR process. Strand separation can be accomplished by any suitable denaturing method including physical, chemical, or enzymatic means. One method of separating the nucleic acid strands involves heating the nucleic acid until it is predominantly denatured (e.g., at least 50%, 60%, 70%, 80%, 90%, or 95% denatured). The heating conditions necessary for denaturing template nucleic acid will depend, e.g., on the buffer salt concentration and the length and nucleotide composition of the nucleic acids being denatured, but typically range from about 85° C. to about 105° C. for a time depending on features of the reaction such as temperature and the nucleic acid length. Denaturation is typically performed from about 2 seconds (in lab-on-chip settings) to 10 minutes.

After the double-stranded nucleic acid is denatured by heat, the reaction mixture is allowed to cool to a temperature that promotes annealing of each primer the nucleic acid sequence comprising the TNAS. The temperature for annealing is usually from about 35° C. to about 65° C. The annealing time is typically from about 1 second to about 1 min. The reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° C. to 80° C.). The extension time is normally from about 5 seconds (in lab-on-chip settings) to about 5 minutes.

Nucleic acid amplification, including PCR, can amplify nucleic acids such as DNA or RNA, including messenger RNA (mRNA). The nucleic acid comprising the TNAS need not be purified; it may be a minor fraction of a complex mixture, such as nucleic acid contained in human cells. DNA or RNA may be extracted from a biological sample such as dermal swabs, cerebrospinal fluid, blood, sputum, bronchio-alveolar lavage, bronchial aspirates, lung tissue, and feces by routine techniques employed by persons known in the art. DNA or RNA also can be extracted from non-biological samples such as air samples, suspicious powders, surface swipes, and rinse products from suspicious solid materials. Nucleic acids can be obtained from any number of sources, such as plasmids, or natural sources including bacteria, yeast, viruses, organelles, or higher organisms such as plants or animals.

In step c) the sample is contacted with the primers and the PCR reagents under reaction conditions that induce primer extension. Typical PCR reagents comprise one or more, and preferably all, the reagents selected form the group consisting of a primer, a nucleic acid, a deoxynucleotide triphosphate and a nucleic acid polymerase. Preferably, the PCR reagents comprise a primer, a nucleic acid, a deoxynucleotide triphosphate and a nucleic acid polymerase.

The PCR reagents may furthermore comprise additives such as 2-mercaptoethanol, e.g. in a concentration of 10 mM, BSA e.g. in a concentration of 1 mg/ml and/or a detergent e.g. in a concentration of 0.5% to 6% (w/v). The detergent can be selected from the group consisting of Triton X-100, Triton X-114, NP-40, Tween20, Tween80 and similar non-ionic detergents.

The salt may be e.g. KCl, at a concentration of e.g. 50 mM, and MgCl₂, at a concentration of e.g. 15 mM.

The buffer may be, e.g., Tris-HCl at a concentration of e.g., 10 mM resulting in a pH of 8.3.

The nucleic acid polymerase may e.g. be a Taq polymerase and may e.g. be present with an activity of 2.5 U.

The deoxynucleotide triphosphates are typically dATP, dCTP, dTTP, and dGTP, or one or more of their analogs. Each of the deoxynucleotide triphosphates are typically each present in the reaction mixture in a concentration within the range of 100-400 μM, such as 150-300 μM.

In a preferred embodiment of the invention, the concentration ratio in the reaction mixture of step c) between the primers of the pair of outer primers and primers of the pair of inner primers is in the range of 10:1-1:100, such as in the range 10:1-1:1, 1:1-1:5, 1:5-1:20, 1:20-1:50, and 1:50-1:100. Preferably, the concentration ratio in the reaction mixture of step c) between the primers of the pair of outer primers and the pair of inner primers is in the range of 1:20-1:50, such as about 1:20 or 1:30. The advantage of keeping the concentration of the outer pair of primers lower that the concentration of the pair of inner primers is that the outer pair of primers will be exhausted during the initial rounds of PCR, i.e. step d) and therefore will not interfere with the nested PCR of step e) utilizing the inner pair of primers.

For example, each of the primers of the inner pair of primers may be present in a concentration of 300 nM and each of the primers of the outer pair of primers may be present in a concentration of 15 nM.

The newly synthesized strands form a double-stranded molecule that can be used in the succeeding steps of the reaction. The steps of strand separation, annealing, and elongation can be repeated as often as needed to produce the desired quantity of amplification products corresponding to the TNAS or a fragment thereof. The limiting factors in the reaction are the amounts of primers, thermostable enzyme, and deoxynucleoside triphosphates present in the reaction. The cycling steps (i.e., denaturation, annealing, and extension) are preferably repeated at least once. For use in detection, the number of cycling steps will depend, e.g., on the nature of the sample. If the sample is a complex mixture of nucleic acids, more cycling steps will be required to amplify the TNAS sufficient for detection. Generally, the cycling steps are repeated at least about 20 times, but may be repeated as many as 40, 60, or even 100 times.

The term “melting temperature” (Tm) of a nucleic acid molecule relates to the temperature at which the nucleic acid molecule hybridized to it complementary counterpart dissociates. The Tm is to be determined experimentally as solvents and salts concentrations have a significant impact on the Tm. The Tm may e.g. be determined using the intercalating DNA dye SYBRGreen™, which is fluorescent when bound the double stranded nucleic acids and which looses its fluorescent properties when released upon dissociation and melting of the double stranded nucleic acids into single stranded nucleic acids (Rasmussen et al.).

The determination of Tm can be used to check the specificity of an amplified product. When the temperature is gradually increased, a sharp decrease in SYBR Green fluorescence is observed as the product undergoes denaturation. Specific products can be distinguished from the non-specific products by the difference in their melting temperatures. A recommended ramping time is 20 minutes for the temperature interval 72-95° C. Optionally, this can be followed by re-annealing at 72° C. for 5-10 min if the samples are to be analyzed e.g. using agarose gel electrophoresis.

Melting curve analysis is typically included in the analysis software of real-time fluorescents detection instruments. When the decrease in SYBR Green fluorescence during the temperature increase is plotted as a negative first derivative (−dF/dT), the temperature of the peak is defined as the Tm, or the melting temperature of the product.

As said, the Tm of the pair of outer primers must be at least 2° C. higher than the Tm of the pair of inner primers.

For example, the Tm of the pair of outer primers may be at least 2° C. higher than the Tm of the pair of inner primers, such as at least 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 15° C., or at least 20° C. higher such as at least 25° C. higher. Preferably, the Tm of the pair of outer primers is at least 10° C. higher than the Tm of the pair of inner primers. For example, the Tm of the pair of outer primers may be e.g. 2-40° C. higher than the Tm of the pair of inner primers, such as 2-5° C. higher, 5-10° C. higher, 10-15° C. higher, 15-20° C. higher, or 20-30° C. higher.

In an important embodiment of the invention, the all the primers are present in the same reaction mixture of step c) and such a nested PCR process can be said to be performed in a single-tube format or a single-mixture format.

Step d) typically includes at least two cycles, such as at least 3, 4, 5, 6, 7, 8, 9, 10, 15, 20, 25, 30, 40, or 50 cycles, such as at least 100 cycles. For example the step d) may include a number of cycles in the range of 2-100, such as in the range of 2-5, 5-10, 10-20, 20-30, 30-40, 40-50, 50-60, 60-70, 70-80, or 80-90, such as in the range of 90-100. Preferably, the number of cycles is in the range of 20-50.

The first denaturation temperature is normally in the range of in the range of 70-105 degrees C., such as in the range of 70-75, 75-80, 80-85, 85-90, 90-95, or 95-100 degrees C., such as in the range of 100-105 degrees C.

The first annealing temperature is typically in the range of in the range of 35-65 degrees C., such as in the range of 35-40, 40-45, 45-50, 50-55, or 55-60 degrees C., such as in the range of 60-65 degrees C.

The first extension temperature may e.g. be in the range of in the range of 40-80 degrees C., such as in the range of 40-45, 45-50, 50-55, 55-60, 60-65, 65-70, or 70-75 degrees C., such as in the range of 75-80 degrees C.

Typically, the first annealing temperature is similar to or lower than the lowest Tm of the outer primer pair and higher than the highest Tm of the inner primer pair,

Similar to step d), step e) typically includes at least two cycles, such as at least 3, 4, 5, 6, 7, 8, 9, 10, 15, 20, 25, 30, 40, or 50 cycles, such as at least 100 cycles. For example the step d) may include a number of cycles in the range of 2-5, such as in the range of 5-10, 10-20, 20-30, 30-40, 40-50, 50-60, 60-70, 70-80, or 80-90, such as in the range of 90-100. Preferably, the number of cycles is in the range of 20-50.

The second denaturation temperature is normally in the range of in the range of 70-105 degrees C., such as in the range of 70-75, 75-80, 80-85, 85-90, 90-95, or 95-100 degrees C., such as in the range of 100-105 degrees C.

The second annealing temperature is typically in the range of in the range of 35-65 degrees C., such as in the range of 35-40, 40-45, 45-50, 50-55, or 55-60 degrees C., such as in the range of 60-65 degrees C.

The second extension temperature may e.g. be in the range of in the range of 40-80 degrees C., such as in the range of 40-45, 45-50, 50-55, 55-60, 60-65, 65-70, or 70-75 degrees C., such as in the range of 75-80 degrees C.

An example of a temperature cycling program according to steps d) and e) of the present invention is shown in Table 4 of Example 2. Both for step d) and/or e), it is envisioned that the annealing temperature and the extension temperature may be the same. This is e.g. the case in cycles of Table 4 related to the outer pair of primers, where both annealing and extension takes place at 63° C.

In a preferred embodiment of the invention, the method furthermore comprises a step f) of analyzing the product of step d and/or step e) to detect the presence of the TNAS. This analysis may be performed in numerous ways, e.g. using fluorescence techniques, electrophoresis, or by means of electrochemical detection such as voltammetry as described herein.

The method may furthermore comprise a step of pre-digesting nucleic acids of the sample. The pre-digestion is preferably performed before to the nucleic acid amplification, but it is additionally envisioned that the pre-digestion also may be performed during the nucleic acid amplification. The nucleic acids of the sample can be pre-digested with an appropriate restriction enzyme. As evident from Table 1 a few commercial restriction enzymes exist that are able to digest DNA at elevated temperature. Pre-digestion will ensure that any background genetic material having the appropriate recognition site will be cleaved at that site. This will in turn impede the unspecific and unwanted amplification of DNA stretches having degenerate primer recognition sites on both sides of the restriction site, thus lowering the background signal prior to the nested PCR. Pre-digestion should only be performed with restriction enzymes having recognition sites lying outside the TNAS. In one embodiment of this invention, the restriction enzyme is PspG I (for reference, see U.S. Pat. No. 5,849,558 “Discovery of and method for cloning and producing the PspGI restriction endonuclease”). This restriction enzyme will digest DNA from B. anthracis at 75° C. to an average fragment size of 2821 bp (calculated as the probability of the sequence of the recognition site occurring, when the chromosomal base composition is known (in this case 30% G-C). Furthermore, this restriction enzyme is undemanding with respect to buffer composition (see Table 2 for buffer composition).

TABLE 1
Commercially available enzymes with an optimum activity at temperatures over
60 degrees Celsius.

| | Recognition | | Optimal | Average fragment size |
|---|---|---|---|---|
| Enzyme* | site | Buffer* | temperature* | B. anthracis (30% G-C): |
| Tsp509 I | AATT | NEBuffer 4: Not | 65° C. | Calculated: 66 bp |
| | TTAA | Recommended | | (pXO1/pXO2≈81/82) |
| | | NEBuffer 1: 100% | | |
| | | NEBuffer 2: 100% | | |
| | | NEBuffer 3: 100% | | |
| PspG I | CCWGG* | NEBuffer 1: 75% | 75° C. | Calculated: 2821 bp |
| | GGWGG | NEBuffer 2: 100% | | (pXO1/pXO2≈1593/2562) |
| | | NEBuffer 3: 50% | | |
| | | NEBuffer 4: 100% | | |
| BstB I | TTCGAA | NEBuffer 1: 75% | 65° C. | Calculated: 2961 bp |
| | AACGAA | NEBuffer 2: 50% | | (pXO1/pXO2≈2929/2709) |
| | | NEBuffer 3: 25% | | |
| | | NEBuffer 4: 100% | | |
| BstE II | GGTNACC | +BSA | 60° C. | Calculated: 16124 bp |
| | CCANTGG | NEBuffer 1: 50% | | (pXO1/pXO2≈18165/15804) |
| | | NEBuffer 2: 75% | | |
| | | NEBuffer 3: 100% | | |
| | | NEBuffer 4: 75% | | |
| Tli I | CTCGAG | + BSA | 75° C. | Calculated: 16124 bp |
| | GAGCTC | NEBuffer 1: 25% | | |
| | | NEBuffer 2: 25% | | |
| | | NEBuffer 3: 100% | | |
| | | NEBuffer 4: 10% | | |

Sequence symbols depict:
A (Adenine), C (Cytosine), G (Guanidine), T (Thymine)
Wobble IUPAC-IUB symbols: * W = A or T
*Product info from New England Biolabs, Inc., WWW.NEB.COM

TABLE 2
Composition of commercially available buffer systems compatible with New
England Biolabs restriction enzymes.

| 1X NEBuffer 1: | 1X NEBuffer 2: | 1X NEBuffer 3: | 1X NEBuffer 4: |
|---|---|---|---|
| 10 mM Bis-Tris-Propane- | 50 mM NaCl | 100 mM NaCl | 50 mM potassium acetate |
| HCl | 10 mM Tris-HCl | 50 mM Tris-HCl | 20 mM Tris-acetate |
| 10 mM MgCl₂ | 10 mM MgCl₂ | 10 mM MgCl₂ | 10 mM magnesium |
| 1 mM dithiothreitol | 1 mM | 1 mM | acetate |
| pH 7.0 @ 25° C. | dithiothreitol | dithiothreitol | 1 mM dithiothreitol |
| | pH 7.9 @ 25° C. | pH 7.9 @ 25° C. | pH 7.9 @ 25° C. |

*Product info from New England Biolabs, Inc., WWW.NEB.COM

In an important embodiment of the invention, at least one primer of the outer primer pair comprises a Tm-increasing component.

Also, both of the primers of the outer primer pair may comprise a Tm-increasing component.

The Tm-increasing component may bind non-specifically to nucleic acids.

The Tm-increasing component may e.g. comprise one or more moieties selected from the group consisting of a modified nucleotide and a minor groove binding agent.

The minor groove binding compound may e.g. comprise a compound selected from the group consisting of a minor groove binding protein, or a coumarin-related compound such as e.g. anthracyclines and alkylators.

The modified nucleotide may e.g. comprise a peptide nucleic acid (PNA) or a locked nucleic acid (LNA).

In a preferred embodiment of the invention, the Tm-increasing component increases the Tm of the primer with at least 1° C. relative to the same primer not comprising the Tm-increasing component. For example, the Tm-increasing component may increase the Tm of the primer with at least 1.5° C., such as at least 2° C., 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., or at least 10° C. lower such as at least 15° C. Also, the Tm-increasing component may increase the Tm of the primer in the range of 1-20° C., such as 1-5° C., 5-10° C., 10-15° C., or 15-20° C.

In an important embodiment of the invention, the second denaturation temperature is at least 1° C. lower than the first denaturation temperature. For example, the second denaturation temperature may at least be 1.5° C. lower than the first denaturation temperature, such as at least 2° C., 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., or at least 10° C. lower such as at least 15° C. lower. Preferably, second denaturation temperature is at least 5° C. lower than the first denaturation temperature. For example, the second denaturing temperature may be 1-30° C. lower than the first denaturation temperature, such as 1-5° C. lower, 5-10° C. lower, 10-15° C. lower, 15-20° C. lower, or 20-30° C. lower.

An important aspect of the invention relates to a method for detection of Bacillus anthracis, the method comprising detecting a TNAS according to the method described herein, the TNAS being specific for the pXO1 or pXO2 plasmid of Bacillus anthracis, wherein the pair of outer primers and the pair of inner primers are selected from the pXO1 or pXO2 plasmid of Bacillus anthracis.

For example, the pair of outer primers and the pair of inner primers may be selected so as to amplify a TNAS related to a gene selected from the group of B. anthracis genes consisting of capA gene, the capB gene, the capC gene, the lef gene.

By the phrase “TNAS related to a gene” it is meant that the TNAS e.g. may be located within the gene of the plasmid or it may be overlapping with the gene on the plasmid.

For amplification or detection of B. anthracis via the capA gene,
a primer of the pair of outer primers may e.g. comprise a nucleic acid sequence selected from the group of SEQ ID NO: 1, SEQ ID NO: 2, a homologous sequence thereof, and a complementary sequence thereof, and
a primer of the pair of inner primers may e.g. comprise a nucleic acid sequence selected from the group of SEQ ID NO: 3, SEQ ID NO: 4, a homologous sequence thereof, and a complementary sequence thereof.

For amplification or detection of B. anthracis via the capA gene,
a primer of the pair of outer primers may e.g. essentially consist of a nucleic acid sequence selected from the group of SEQ ID NO: 1, SEQ ID NO: 2, a homologous sequence thereof, and a complementary sequence thereof, and
a primer of the pair of inner primers may e.g. essentially consist of a nucleic acid sequence selected from the group of SEQ ID NO: 3, SEQ ID NO: 4, a homologous sequence thereof, and a complementary sequence thereof.

For detection of B. anthracis via the capA gene, the pair of outer primers may e.g. comprise SEQ ID NOs: 1 and/or 2 or their complementary sequences. Also, for the detection of B. anthracis via the capA gene, the pair of inner primers may e.g. comprise SEQ ID NOs: 3 and/or 4 or their complementary sequences.

In a preferred embodiment of the invention, for detection of B. anthracis via the capA gene, the pair of outer primers comprises SEQ ID NOs: 1 and 2 and the pair of inner primers comprise SEQ ID NOs: 3 and 4. Alternatively, the pair of outer primers comprise the complementary sequences of SEQ ID NOs: 1 and 2 and the pair of inner primers comprise the complementary sequences of SEQ ID NOs: 3 and 4.

Also, for detection of B. anthracis via the capA gene, the pair of outer primers may e.g. essentially consist of SEQ ID NOs: 1 and/or 2 or their complementary sequences. Also, for the detection of B. anthracis via the capA gene, the pair of inner primers may e.g. essentially consist of SEQ ID NOs: 3 and/or 4 or their complementary sequences.

In a preferred embodiment of the invention, for detection of B. anthracis via the capA gene, the pair of outer primers essentially consists of SEQ ID NOs: 1 and 2 and the pair of inner primers essentially consist of SEQ ID NOs: 3 and 4. Alternatively, the pair of outer primers essentially consist of the complementary sequences of SEQ ID NOs: 1 and 2 and the pair of inner primers essentially consist of the complementary sequences of SEQ ID NOs: 3 and 4.

According to the present invention, the term “essentially consist” or “essentially consisting” is meant that one or more of the mentioned nucleic acids are essential and necessary for the detection of B. anthracis. However, beside the mentioned sequences, the primers may furthermore comprise e.g. Tm-increasing components and/or various markers for detection purposes such as e.g. fluorescent or electrochemical markers.

Another aspect of the present invention relates to kit comprising a pair of outer primers and a pair of inner primer, the melting temperature (Tm) of the pair of outer primers being higher than the Tm of the pair of inner primers.

The kit may furthermore comprise one or more PCR reagents comprise one or more reagents selected form the group consisting of a primer, a nucleic acid, a deoxynucleotide triphosphate and a nucleic acid polymerase. For example the kit furthermore comprises a nucleic acid, a deoxynucleotide triphosphate and a nucleic acid polymerase.

The kit may furthermore comprise additives such as 2-mercaptoethanol, e.g. in an amount the results in a concentration of 10 mM when the kit is used. The kit may furthermore comprise additives such as BSA, e.g. in an amount the results in a concentration of 1 mg/ml when the kit is used. The kit may furthermore comprise additives such as a detergent e.g. in an amount the results in a concentration of 0.5% to 6% (w/v) when the kit is used. The detergent may e.g. be selected from the group consisting of Triton X-100, Triton X-114, NP-40, Tween20, Tween80 and similar non-ionic detergents.

In a preferred embodiment of the invention, at least one primer of the outer primer pair comprises a Tm-increasing component as defined herein. Preferably, both of the primers of the outer primer pair comprise a Tm-increasing component. The primers of the outer pair may comprise identical Tm-increasing components. That is to say, both the first primer of the outer primer pair and the second primer of the outer primer pair may comprise a Tm-increasing component, which e.g. is a minor groove binding protein.

Alternatively, the primers of the outer pair may comprise different Tm-increasing components. That is to say, the first primer of the outer primer pair may e.g. comprise a Tm-increasing component which is a modified nucleotide and the second primer of the outer primer pair may e.g. comprise a Tm-increasing component, which is a minor groove binding protein.

In an important embodiment of the invention, the kit is for detection of Bacillus anthracis, the kit comprising a pair of outer primers and a pair of inner primer, the melting temperature (Tm) of the pair of outer primers being higher than the Tm of the pair of inner primers, wherein the pair of outer primers and the pair of inner primers are selected from the pXO1 or pXO2 plasmid of Bacillus anthracis.

For example, in the kit the pair of outer primers and the pair of inner primers may be selected so as to amplify a TNAS related to a gene selected from the group of B. anthracis genes consisting of capA gene, the capB gene, the capC gene, the lef gene.

In a preferred embodiment of the invention, the pair of outer primers may e.g. comprise SEQ ID NOs: 1 and/or 2 or their complementary sequences. Also, for the detection of B. anthracis via the capA gene, the pair of inner primers may e.g. comprise SEQ ID NOs: 3 and/or 4 or their complementary sequences.

For example, in the kit the pair of outer primers comprises SEQ ID NOs: 1 and 2 and the pair of inner primers comprise SEQ ID NOs: 3 and 4. Alternatively, the pair of outer primers comprise the complementary sequences of SEQ ID NOs: 1 and 2 and the pair of inner primers comprise the complementary sequences of SEQ ID NOs: 3 and 4.

Also, in the kit the pair of outer primers may e.g. essentially consist of SEQ ID NOs: 1 and/or 2 or their complementary sequences. Also, for the detection of B. anthracis via the capA gene, the pair of inner primers may e.g. essentially consist of SEQ ID NOs: 3 and/or 4 or their complementary sequences.

In a preferred embodiment of the invention, the pair of outer primers of the kit essentially consists of SEQ ID NOs: 1 and 2 and the pair of inner primers of the kit essentially consist of SEQ ID NOs: 3 and 4. Alternatively, the pair of outer primers of the kit essentially consists of the complementary sequences of SEQ ID NOs: 1 and 2 and the pair of inner primers of the kit essentially consist of the complementary sequences of SEQ ID NOs: 3 and 4.

A further aspect of the invention relates to an analysis system for detection of a microorganism, the analysis system comprising a pair of outer primers and a pair of inner primer, the melting temperature (Tm) of the pair of outer primers being higher than the Tm of the pair of inner primers.

Typically the analysis system is an automated analysis system and it may comprise a component selected from the group consisting of a lateral flow device, a biochip, and a microarray. A presently preferred analysis system is described in the co-pending PCT Application No. TTTTTTTTT with the title “Method, chip, device and integrated system for detection of biological particles”, which is incorporated herein by reference.

A special aspect of the present invention relates to methods of and kits for a sensitive and highly specific identification of Bacillus anthracis in a biological sample or in a non-biological sample. Primers and probes for detecting B. anthracis are provided by the invention, as are kits containing such primers and probes. Methods of the invention can be used to rapidly identify B. anthracis DNA from specimens for diagnosis of B. anthracis infection and to identify hoax cases of B. anthracis. Using specific primers and probes, the methods include amplifying and monitoring the development of specific amplification products using detection systems based on either voltammetric analysis of electrochemically active probes or detection of fluorescence.

Though described in the context of B. anthracis detection, it is envisioned that the all of the features mentioned herein are generally applicable for the amplification and/or detection of any TNAS, or microorganisms or vira comprising a TNAS.

Bacillus anthracis, the causative agent of Anthrax, is a large, aerobic, Gram-positive, spore-forming, non-motile Bacillus. Spores are formed in culture, in the soil, and in the tissues and exudates of dead animals when there is limited access to nutrients, and subsequently not in the blood or tissues of living animals. Spores can remain viable in soil for decades. The bacterium ordinarily produces a zoonotic disease in domesticated and wild animals such as goats, sheep, cattle, horses, and swine. Humans become infected by the cutaneous route (direct contact with diseased animals, industrial work with hides, wool, brushes, or bone meal), by inhalation (Woolsorter's disease), or by ingestion (meat from diseased animals).

Anthrax endospores do not divide, have no measurable metabolism, and are very resistant to drying out, heat, ultraviolet light, gamma radiation, and many disinfectants. All known anthrax virulence genes are expressed by the vegetative form of B. anthracis upon germination of spores within the body of the host. Endospores introduced into the body by abrasion, inhalation, or ingestion are phagocytized by macrophages and carried to regional lymph nodes. The endospores germinate inside the macrophages and become vegetative bacteria; the vegetative bacteria are then released from the macrophages, multiply in the lymphatic system, and enter the bloodstream until there is as many as 10⁷ to 10⁸ organisms/ml blood, causing massive septicemia. Once they have been released from the macrophages, there is no evidence that an immune response is initiated against vegetative bacilli. Anthrax bacilli express virulence factors, including toxin and capsule polypeptides. The resulting toxemia has systemic effects that lead to the death of the host.

The major virulence factors of B. anthracis are encoded on two virulence plasmids, pXO1 (GenBank Accession Nos. AF065404, AE011190, NC001496, and NC003980) and pXO2 (GenBank Accession Nos. AF188935, AE011191, and NC003981). The toxin-bearing plasmid, pXO1, is 181.6 kilobases (kb) in size and comprises the genes that code for the secreted exotoxins. The toxin gene complex is composed of protective antigen (PA), lethal factor (LF), and edema factor (EF). The three exotoxin components combine to form two binary toxins. Edema toxin consists of EF, which is a calmodulin-dependent adenylate cyclase, and PA, the binding moiety that permits entry of the toxin into the host cell. Increased cellular levels of CAMP upset water homeostasis and are believed to be responsible for the massive edema seen in cutaneous anthrax. Edema toxin inhibits neutrophile function in vitro and neutrophile function is impaired in patients with cutaneous anthrax infection. Lethal toxin consists of LF, which is a zinc metalloprotease that inactivates mitogen-activated-protein kinase kinase (MAPKK) in vitro, and PA, which acts as the binding domain. Lethal toxin stimulates the macrophages to release tumor necrosis factor-α and interleukin-β, which are partly responsible for sudden death in systemic anthrax. The capsule-bearing plasmid, pXO2, is 96.2 kb in size and comprises three genes (capA, capB, and capC,) involved in the synthesis of the poly-D-glutamic capsule.

The exotoxins are thought to inhibit the immune response mounted against infection, whereas the capsule inhibits phagocytosis of vegetative anthrax bacilli. The expression of all known major virulence factors is regulated by host-specific factors such as elevated temperature (>37° C.) and carbon dioxide concentration (>5%) and by the presence of serum components. Both plasmids are required for full virulence and the loss of either one result in an attenuated strain. Historically, anthrax vaccines were made by rendering virulent strains free of one or both plasmids. By way of example, Pasteur is an avirulent pXO2-carrying strain that is encapsulated but does not express exotoxin components, while Sterne is an attenuated strain that carries pXO1 and can synthesize exotoxin components but does not have a capsule. The Sterne strain seems to provide the best protection, as this strain comprises the pXO1 plasmid and thus is able to synthesize the Protective Antigen, a protein that gives the optimum immunogenic response in vaccines.

In a special aspect of the invention, a method is provided for detecting the presence or absence of B. anthracis in a biological sample from an individual or in a non-biological sample. Using a polymerase having additional 5′-3′ exonuclease activity, the method to detect B. anthracis includes performing at least one cycling step, which includes an amplifying step and a hybridizing step. The amplifying step includes contacting the sample with a pair of capA primers to produce a capA amplification product if a B. anthracis capA nucleic acid molecule is present in the sample.

In another special aspect of the invention, there is provided a method for further amplifying the previously capA amplification product in the same reaction by contacting said capA amplification product with another pair of nested capA primers to generate another capA amplification product if a B. anthracis capA nucleic acid molecule is present in the sample.

Primers useful in the present invention include oligonucleotides capable of acting as a point of initiation of nucleic acid synthesis within B. anthracis capA or Lef.

Functional isolation of the two capA primer pairs is achieved by designing primer pairs with different Tₘ values and by using different molar amounts of outer and nested capA primers, the molar ratio being between 1/10 and 1/500. The outer capA primers are designed to have a Tₘ 10° C. higher than the nested capA primers. The probe is designed to have a Tₘ 10° C. higher than the Tₘ of the nested capA primers, and to hybridize as little as possible with any of the capA primers, nor with copies of itself.

The hybridizing step includes contacting the sample with a capA probe. The capA probe is typically labeled with an electrochemically active marker such as a metallocene, more specifically ferrocene. In solution, the accumulated digested probe will be distinguished from undigested probe due to its different electrochemical activity; the method therefore further includes the detection of the presence or absence of a probe-specific voltage peak using a detection system based on voltammetric analysis of electrochemical activity. The presence of a probe-specific voltage peak is usually indicative of the presence of B. anthracis in the sample, while the absence of a probe-specific voltage peak is usually indicative of the absence of B. anthracis in the sample.

Alternatively, the capA probe is labeled at the most 5′ base with a fluorescent dye and at the most 3′ base with a fluorescent quencher dye which hybridizes within the target DNA; said labeled oligonucleotide probe being susceptible to 5′-3′ exonuclease degradation by said polymerase to produce fragments that can be detected by fluorogenic methods. The presence of fluorescence is usually indicative of the presence of B. anthracis in the sample, while the absence of fluorescence is usually indicative of the absence of B. anthracis in the sample.

Alternatively or additionally, the amplifying step can include contacting the sample with a pair of Lef primers to produce an amplification product if a B. anthracis Lef nucleic acid molecule is present in the sample.

In another special aspect of the invention, there is provided a method for further amplifying the previously Lef amplification product in the same reaction by contacting said Lef amplification product with another pair of nested Lef primers to generate another Lef amplification product if a B. anthracis Lef nucleic acid molecule is present in the sample.

Functional isolation of the two Lef primer pairs is achieved by designing primer pairs with different Tₘ values and by using different molar amounts of outer and nested Lef primers, the molar ratio being between 1/10 and 1/500. The outer Lef primers are designed to have a Tₘ 10° C. higher than the nested Lef primers. The probe is designed to have a Tₘ 10° C. higher than the Tₘ of the nested Lef primers, and to hybridize as little as possible with any of the Lef primers, nor with copies of itself.

The hybridizing step includes contacting the sample with a Lef probe. The Lef probe is typically labeled with an electrochemically active marker such as a metallocene, more specifically ferrocene. In solution, the accumulated digested probe will be distinguished from undigested probe due to its different electrochemical activity; the method therefore further includes the detection of the presence or absence of a probe-specific voltage peak using a detection system based on voltammetric analysis of electrochemical activity. The presence of a probe-specific voltage peak is usually indicative of the presence of B. anthracis in the sample, while the absence of a probe-specific voltage peak is usually indicative of the absence of B. anthracis in the sample.

Alternatively, the Lef probe is labeled at the most 5′ base with a fluorescent dye and at the most 3′ base with a fluorescent quencher dye which hybridizes within the target DNA; said labeled oligonucleotide probe being susceptible to 5′-3′ exonuclease degradation by said polymerase to produce fragments that can be detected by fluorogenic methods. The presence of fluorescence is usually indicative of the presence of B. anthracis in the sample, while the absence of fluorescence is usually indicative of the absence of B. anthracis in the sample. The methods to detect B. anthracis using capA and/or Lef can be performed individually, sequentially or simultaneously.

Suitable electrochemically active markers include those comprising metallo-carbocyclic pi complexes, that is organic complexes with partially or fully delocalized pi electrons. Suitable markers include those comprising sandwich compounds, in which two carbocyclic rings are parallel, and also bent sandwiches (angular compounds) and monocyclopentadienyls.

Preferably, the electrochemically active markers are metallocenyl labels. More preferably they are ferrocenyl labels. A representative label for the probe is ferrocenyl and metallocenyl, more advantageously N-substituted ferrocene or metallocene carboxamides. The ferrocene or metallocene ring, which constitutes the labeling moiety, may be un-substituted. Additional corresponding suitable electrochemically active markers are known in the art.

In a special aspect, the detecting step includes differential pulse voltammetry. The voltammogram traces for the two markers should have probe-specific voltage peaks that are resolvable from each other. In another special aspect, the detecting step includes quantitating the probe-specific voltage peaks. In yet another special aspect, the detecting step can be performed after each cycling step (e.g., in real-time).

In yet another special aspect, the detecting step includes exciting the sample at a wavelength absorbed by the fluorescent moiety and visualizing and/or measuring the wavelength emitted. In another special aspect, the detecting step includes quantitating the fluorescence. In yet another special aspect, the detecting step can be performed after each cycling step (e.g., in real-time).

Generally, the presence of probe-specific voltage peaks (as measured by differential pulse voltammetry) within 45 cycles (e.g., 20, 25, 30, 35, or 40 cycles) indicates the presence of a B. anthracis infection in the individual. In addition, determining the melting temperature between the capA probe and the capA amplification product or, similarly, between the Lef probe and the Lef amplification product, respectively, can confirm the presence or absence of the B. anthracis.

Alternatively or additionally, the presence of fluorescence within 45 cycles (e.g., 20, 25, 30, 35, or 40 cycles) indicates the presence of a B. anthracis infection in the individual.

Representative biological sample include dermal swabs, cerebrospinal fluid, blood, sputum, bronchio-alveolar lavage, bronchial aspirates, lung tissue, and urine. Non-biological samples include powders, air samples, surface swipes, and rinse products from solid materials. Biological or non-biological samples can be cultured. The culture then can be evaluated for B. anthracis using the methods of the invention.

In addition, the cycling step can be performed on a control sample. A control sample can include the same portion of the B. anthracis capA nucleic acid molecule. Alternatively, a control sample can include a nucleic acid molecule other than a B. anthracis capA nucleic acid molecule. Cycling steps can be performed on such a control sample using a pair of control primers and a control probe. The control primers and probe are other than capA primers and capA probe. One or more amplifying steps produce a control amplification product. Each of the control probes hybridizes to the control amplification product.

In another special aspect of the invention, there are provided articles of manufacture, or kits. Kits of the invention can include a pair of capA primers, and a capA probe, and a donor and corresponding acceptor fluorescent moieties. For example, the first capA primer provided in a kit of the invention can have the sequence 5′-GGC GAA ACA TGA CGA AAA AC-3′ (SEQ ID NO:1) and the second capA primer can have the sequence 5′-CCT CGT TAT GTA GCA ATC GTA TTA C-3′ (SEQ ID NO:2). The capA probe provided in a kit of the invention can have the sequence 5′-CCA TCG TCA TCG TCA AT-3′ (SEQ ID NO:13).

For example, the first nested capA primer provided in a kit of the invention can have the sequence 5′-TTA CGT GAC GTC CCA TC-3′ (SEQ ID NO:3) and the second nested capA primer can have the sequence 5′-TGC GAC ATG GGT ACA AC-3′ (SEQ ID NO:4).

Articles of manufacture of the invention can further or alternatively include a pair of Lef primers, a pair of page probes, and a donor and corresponding acceptor fluorescent moieties. For example, the first Lef primer provided in a kit of the invention can have the sequence 5′-AAA AGG TAA CAA ATT ACT TAG TTG ATG G-3′ (SEQ ID NO:6), and the second Lef primer can have the sequence 5′-CGA AGT TAA ATT ACT CCC TTC TTC CTT-3′ (SEQ ID NO:7). The Lef probe provided in a kit of the invention can have the sequence 5′-TCA AAA GGT GTA GAA TTA AGG-3′ (SEQ ID NO:14)

For example, the first nested Lef primer provided in a kit of the invention can have the sequence 5′-GGG TTA TAT GTT CCA GAA TC-3′ (SEQ ID NO:8) and the second nested Lef primer can have the sequence 5′-GTA ACT AAA TCA GAT TGG TTC T-3′ (SEQ ID NO:9).

The article of manufacture can include a package insert having instructions thereon for using the primers, and probes to detect the presence or absence of B. anthracis in a sample.

In yet another special aspect of the invention there is provided a method for detecting the presence or absence of B. anthracis in a biological sample from an individual or in a non-biological sample. Such a method includes performing at least one cycling step. A cycling step can include an amplifying step and a hybridizing step. Generally, an amplifying step includes contacting the sample with a pair of capA primers to produce a capA amplification product if a B. anthracis capA nucleic acid molecule is present in the sample. Generally, a hybridizing step includes contacting the sample with a capA probe. Such a capA probe is usually labeled with a fluorescent dye and with a fluorescent quencher dye. The method further includes detecting the presence or absence of fluorescence. The presence or absence of fluorescence is indicative of the presence or absence of B. anthracis in said sample. In addition to the capA primers/probe described herein, this method also can be performed using Lef and/or Lef primers/probe.

In another special aspect of the invention, there is provided a method for detecting the presence or absence of B. anthracis in a biological sample from an individual or in a non-biological sample. Such a method includes performing at least one cycling step. A cycling step can include an amplifying step and a dye-binding step. An amplifying step generally includes contacting the sample with a pair of capA primers to produce a capA amplification product if a B. anthracis capA nucleic acid molecule is present in the sample. A dye-binding step generally includes contacting the capA amplification product with a double-stranded DNA binding dye. The method further includes detecting the presence or absence of binding of the double-stranded DNA binding dye into the amplification product. According to the invention, the presence of binding is typically indicative of the presence of B. anthracis in the sample, and the absence of binding is typically indicative of the absence of B. anthracis in the sample. Such a method can further include the steps of determining the melting temperature between the capA amplification product and the double-stranded DNA binding dye. Generally, the melting temperature confirms the presence or absence of B. anthracis. Representative double-stranded DNA binding dyes include SYBRGreen™, SYBRGold™, and ethidium bromide.

The details of one or more embodiments of the invention are set forth in the accompanying drawings and the description below. Other features, objects, and advantages of the invention will be apparent from the drawings and detailed description, and from the claims.

A real-time assay for detecting B. anthracis in a biological sample or in a non-biological sample that is more sensitive and specific than existing assays is described herein. Primers and probes for detecting B. anthracis infections and articles of manufacture containing such primers and probes are provided by the invention. The increased sensitivity of real-time PCR for detection of B. anthracis compared to other methods, as well as the improved features of real-time PCR including sample containment and real-time detection of the amplified product, make feasible the implementation of this technology for routine diagnosis of B. anthracis infections in the clinical laboratory.

The invention provides methods to detect B. anthracis by amplifying, for example, a portion of the B. anthracis capA, or Lef nucleic acid. B. anthracis nucleic acids other than those exemplified herein (e.g., other than capA or lef) also can be used to detect B. anthracis in a sample and are known to those of skill in the art. The nucleic acid sequence of B. anthracis capA (encoding encapsulation protein A) and Lef (encoding lethal factor) are available (see, for example, GenBank Accession Nos. M24150, M29081, and M30210). Specifically, primers and probes to amplify and detect B. anthracis capA nucleic acid molecules are provided by the invention, as are primers and probes to amplify and detect B. anthracis Lef nucleic acid molecules and B. anthracis Lef nucleic acid molecules.

Primers that amplify a B. anthracis nucleic acid molecule, e.g., B. anthracis capA or Lef can be designed using, for example, a computer program such as VectorNTI™ (Informax, Inc., Frederick, Md.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 15 to 30 nucleotides in length.

Designing oligonucleotides to be used as hybridization probes can be performed in a manner similar to the design of primers. In addition, probes can be designed to hybridize to targets that contain a polymorphism or mutation, thereby allowing differential detection of B. anthracis strains based on either absolute hybridization of different pairs of probes corresponding to the particular B. anthracis strain to be distinguished or differential melting temperatures between, for example, members of a pair of probes and each amplification product corresponding to a B. anthracis strain to be distinguished. As with oligonucleotide primers, oligonucleotide probes usually have similar melting temperatures, and the length of each probe must be sufficient for sequence-specific hybridization to occur but not so long that fidelity is reduced during synthesis. Oligonucleotide probes are generally 15 to 30 nucleotides in length.

If the B. anthracis template nucleic acid is double-stranded, it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominantly denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed from about 2 seconds (in lab-on-chip settings) to 10 minutes.

After the double-stranded nucleic acid is denatured by heat, the reaction mixture is allowed to cool to a temperature that promotes annealing of each primer to its target sequence on the B. anthracis nucleic acid. The temperature for annealing is usually from about 35° C. to about 65° C. Annealing times can be from about 1 second (in lab-on-chip settings) to about 1 min. The reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° C. to 80° C.). Extension times can be from about 5 seconds (in lab-on-chip settings) to about 5 minutes.

PCR assays can employ B. anthracis nucleic acid such as DNA or RNA, including messenger RNA (mRNA). The template nucleic acid need not be purified; it may be a minor fraction of a complex mixture, such as B. anthracis nucleic acid contained in human cells. DNA or RNA may be extracted from a biological sample such as dermal swabs, cerebrospinal fluid, blood, sputum, bronchio-alveolar lavage, bronchial aspirates, lung tissue, and feces by routine techniques employed by persons known in the art. If B. anthracis is present, DNA or RNA also can be extracted from non-biological samples such as air samples, suspicious powders, surface swipes, and rinse products from suspicious solid materials. Nucleic acids can be obtained from any number of sources, such as plasmids, or natural sources including bacteria, yeast, viruses, organelles, or higher organisms such as plants or animals.

The oligonucleotide primers are combined with PCR reagents under reaction conditions that induce primer extension. For example, chain extension reactions generally include 50 mM KCl, 10 mM Tris-HCl (pH 8.3), 15 mM MgCl₂, 0.5-1.0 μg denatured template DNA, 50 pmoles of each oligonucleotide primer, 2.5 U of Taq polymerase, and 10% DMSO). The reactions usually contain 150 to 300 μM each of dATP, dCTP, dTTP, dGTP, or one or more analogs thereof.

The newly synthesized strands form a double-stranded molecule that can be used in the succeeding steps of the reaction. The steps of strand separation, annealing, and elongation can be repeated as often as needed to produce the desired quantity of amplification products corresponding to the TNAS. The limiting factors in the reaction are the amounts of primers, thermo stable enzyme, and nucleoside triphosphates present in the reaction. The cycling steps (i.e., denaturation, annealing, and extension) are preferably repeated at least once. For use in detection, the number of cycling steps will depend, e.g., on the nature of the sample. If the sample is a complex mixture of nucleic acids, more cycling steps will be required to amplify the target sequence sufficient for detection. Generally, the cycling steps are repeated at least about 20 times, but may be repeated as many as 40, 60, or even 100 times.

DPV technology (see, for example, U.S. Pat. No. 4,083,754) is used for both qualitative and quantitative analysis. The method take advantage of computer timing to repeatedly sample current signals at two points relative to the time of application of a voltage signal to the electrode. The difference between the two current values is plotted as a function of the applied DC potential. The resultant is peaks, corresponding to the electro-activity of the species in the electrochemical cell.

Electrochemical methods, when compared to the conventional methods for DNA analysis (e.g., fluorescence), are straightforward and sensitive and do not require sophisticated instrumentation. Consequently, they are suitable for the development of inexpensive and portable devices for detection and disease diagnoses. Typically, the DNA hybridization is detected by electrochemical reactions of a redox marker or reporter on the target (e.g., ferrocene).

The presence of B. anthracis is generally being detected by culturing the organism. The success of culturing B. anthracis from clinical specimens depends in part upon the B. anthracis infection and hence, the source of the specimen. Cultures from skin lesions associated with the cutaneous form of the disease can exhibit 60-65% sensitivity, and often are not diagnostically useful. Generally, cultures from blood exhibit a high sensitivity due to the extremely large number of circulating B. anthracis organisms. Patients with systemic disease, however, often expire within the time necessary for blood cultures to become positive. Other biological samples including sputum and cerebrospinal fluid (CSF) can also be used for culture, but the identification may come too late to initiate effective antibiotic therapy. In the case of gastrointestinal anthrax, cultures from stool samples also can be used.

In addition, serologic tests including enzyme-linked immunosorbent assays (ELISAs) for the detection of B. anthracis have been reported. The sensitivity of ELISA to detect serum antibodies to various targets in B. anthracis including the encapsulation protein, protective antigen, lethal factor, and edema factors varies between 26% and 100%, depending upon the target and study.

Direct detection of B. anthracis from clinical specimens or suspicious substances using stains is possible and can allow a presumptive identification of B. anthracis. With Gram staining, the cells are visualized as large Gram-positive encapsulated rods. The success of this staining technique depends upon the presence of a sufficient number of organisms. Typically, staining techniques provide a presumptive identification of B. anthracis and a definitive diagnosis generally requires further evaluation.

Conventional PCR methods also have been used to detect B. anthracis. B. anthracis and other members of the B. cereus group, however, exhibit a high degree of genomic homology, making detection and differentiation by PCR difficult. Conventional PCR-based amplification is generally followed by transfer of the amplification products to a solid support and detection using a labeled probe (e.g., a Southern or Northern blot). These methods are labor intensive and frequently require more than one day to complete. Additionally, the manipulation of amplification products for the purpose of detection (e.g., by blotting) increases the risk of carry-over contamination and false positives. By using commercially available real-time PCR instrumentation (e.g., DNA Engine Opticon™ MJ research, Reno, Nev.), PCR amplification and detection of the amplification product can be combined in a single closed cuvette with dramatically reduced cycling time. Since detection occurs concurrently with amplification, the real-time PCR methods prevent the need for manipulation of the amplification product, and therefore diminish the risk of cross-contamination between amplification products. Real-time PCR greatly reduces turn-around time and is an attractive alternative to conventional PCR techniques in the clinical laboratory.

The present invention provides methods for detecting the presence or absence of B. anthracis in a biological sample from an individual or in a non-biological sample. Methods provided by the invention avoid problems of sample contamination, false negatives, and false positives. The methods include performing at least one cycling step that includes amplifying a B. anthracis portion of a capA and/or Lef nucleic acid molecule from a sample using a pair of capA and/or Lef primers, respectively. Each of the capA or Lef primers anneals to a target within or adjacent to a B. anthracis capA or Lef nucleic acid molecule, respectively, such that at least a portion of each amplification product contains nucleic acid sequence corresponding to capA or lef, respectively. More importantly, the amplification product should contain the nucleic acid sequences that are complementary to the capA or Lef probes, respectively. The capA and/or Lef amplification product is produced provided that B. anthracis nucleic acid is present. Each cycling step further includes contacting the sample with a pair of capA and/or Lef probes. According to the invention, the capA and Lef probes are typically labeled with an electrochemically active marker such as a metallocene, more specifically ferrocene. In solution, the accumulated digested probe will be distinguished from undigested probe due to its different electrochemical activity; the method therefore further includes the detection of the presence or absence of a probe-specific voltage peak (PSVP) using a detection system based on voltametric analysis of electrochemical activity. The presence of a probe-specific voltage peak is usually indicative of the presence of B. anthracis in the sample, while the absence of a probe-specific voltage peak is usually indicative of the absence of B. anthracis in the sample.

Alternatively, the capA and Lef probes are labeled at the most 5′ base with a fluorescent dye and at the most 3′ base with a fluorescent quencher dye which hybridizes within the target DNA; said labeled oligonucleotide probe being susceptible to 5′-3′ exonuclease degradation by said polymerase to produce fragments that can be detected by fluorogenic methods.

Each cycling step includes an amplification step and a hybridization step, and each cycling step is usually followed by respectively a PSVP or FRET detection step. Multiple cycling steps are performed, preferably in a thermocycler. Methods of the invention can be performed using one or more of the capA and/or Lef primer and probe sets to detect the presence of B. anthracis. Alternatively, methods of the invention can be performed simultaneously with each of the capA and Lef primer and probe sets to detect the presence of virulent forms of B. anthracis. Detection of PSVP or FRET signal in one or more, but not all, of the capA and Lef reactions indicates the presence of a B. anthracis strain lacking one or both of the virulence plasmids. Methods of the invention, therefore, also are useful for detecting false claims of anthrax.

As used herein, “amplifying” refers to the process of synthesizing nucleic acid molecules that are complementary to one or both strands of a template nucleic acid molecule (e.g., B. anthracis capA or Lef nucleic acid molecules). Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleoside triphosphates, a DNA polymerase enzyme (e.g., Platinum® Taq) and an appropriate buffer and/or co-factors for optimal activity of the polymerase enzyme (e.g., MgCl₂ and/or KCl).

If amplification of B. anthracis nucleic acid occurs and an amplification product is produced, the step of hybridizing results in a detectable signal based upon PSVP or FRET of the degraded probe. As used herein, “hybridizing” refers to the annealing of probes to an amplification product. Hybridization conditions typically include a temperature that is below the melting temperature of the probes but that avoids non-specific hybridization of the probes.

Generally, the presence of PSVP or FRET indicates the presence of B. anthracis in the sample, and the absence of PSVP or FRET indicates the absence of B. anthracis in the sample. However, inadequate specimen collection, transportation delays, inappropriate transportation conditions, or use of certain collection swabs (calcium alginate or aluminum shaft) are all conditions that can affect the success and/or accuracy of a test result. Using the methods disclosed herein, detection of PSVP or FRET within 50 nested cycling steps is indicative of a B. anthracis infection or contamination.

Representative biological samples that can be used in practicing the methods of the invention include dermal swabs, nasal swaps, cerebrospinal fluid, blood, sputum, bronchio-alveolar lavage, bronchial aspirates, lung tissue, and feces. Collection and storage methods of biological samples are known to those of skill in the art. Biological samples can be processed (e.g., by nucleic acid extraction methods and/or kits known in the art) to release B. anthracis nucleic acid or in some cases, the biological sample can be contacted directly with the PCR reaction components and the appropriate oligonucleotides.

Non-biological samples such as air samples (filtered or non-filtered), powders, and surface swipes and rinse products from suspicious materials also can be examined for the detection of B. anthracis. For example, a powder can be dissolved in a solvent such as water, and the methods of the invention can be performed on varying dilutions (e.g., 1:10, 1:100, or 1:1000) of the resulting solution. Water can be added to a collection vial of an air sample collection device and assayed using methods of the invention, or alternatively, a filter on an air sample collection device can be rinsed and assayed. In addition, a solid material (e.g., paper) can be swiped or rinsed for the purpose of detecting B. anthracis, and a non-turbid solution produced. Dilutions of such a surface swipe or rinse can be used in a real-time amplification reaction of the invention.

Biological or non-biological samples can be cultured in a medium suitable for growth of B. anthracis. The culture media then can be assayed for the presence or absence of B. anthracis using the methods of the invention as described herein. For example, samples arriving at a clinical laboratory for detection of B. anthracis using the methods of the invention can be in the form of a liquid culture that had been inoculated with a biological sample from an individual or with a non-biological sample.

Melting curve analysis is an additional step that can be included in a cycling profile. Melting curve analysis is based on the fact that DNA melts at a characteristic temperature called the melting temperature (Tₘ), which is defined as the temperature at which half of the DNA duplexes have separated into single strands. The melting temperature of a DNA depends primarily upon its nucleotide composition. Thus, DNA molecules rich in G and C nucleotides have a higher Tm than those having an abundance of A and T nucleotides. By detecting the temperature at which signal is lost, the melting temperature of probes can be determined. Similarly, by detecting the temperature at which signal is generated, the annealing temperature of probes can be determined. The melting temperature(s) of the capA or Lef probes from the respective amplification product can confirm the presence or absence of B. anthracis in the sample.

Within each thermocycler run, control samples are cycled as well. In the positive control samples B. anthracis nucleic acid control template can be amplified (other than capA or Lef) using, for example, control primers and control probes. Positive control samples can comprise, for example, a plasmid construct containing B. anthracis capA or Lef nucleic acid molecule. Such a plasmid control can be amplified internally (e.g., within the sample) or in a separate sample run side-by-side with the samples to be examined. Each thermocycler run should also include a negative control that, for example, lacks B. anthracis template DNA. Such non-template controls are indicators of the success or failure of the amplification, hybridization and/or fluorescence reaction. Therefore, control reactions can readily determine, for example, the ability of primers to anneal with sequence-specificity and to initiate elongation, as well as the ability of the probe to hybridize with sequence-specificity and for fluorescence to occur.

Standard laboratory containment practices and procedures are desirable when performing methods of the invention. Containment practices and procedures include, but are not limited to, separate work areas for different steps of a method, containment hoods, barrier filter pipette tips and dedicated air displacement pipettes. Consistent containment practices and procedures by personnel are necessary for accuracy in a diagnostic laboratory handling clinical samples.

Conventional PCR methods in conjunction with fluorescence technology can be used to practice the methods of the invention. In one special embodiment, a DNA Engine Opticon™ instrument is used (a detailed description of the DNA Engine Opticon™ System and real-time and on-line monitoring of PCR can be found at http://www.mjresearch.com/html/instruments/opticon/index.html).

The DNA Engine Opticon™ can be operated using a PC workstation and can utilize a Windows XP operating system. The software can display the fluorescence signals in real-time immediately after each measurement. Fluorescent acquisition time is 10-100 milliseconds (msec). After each cycling step, a quantitative display of fluorescence vs. cycle number can be continually updated for all samples. The data generated can be stored for further analysis.

An amplification product can be detected using a double-stranded DNA binding dye such as a fluorescent DNA binding dye (e.g., SYBRGreenI® or SYBRGold® (Molecular Probes)). Upon interaction with the double-stranded nucleic acid, such fluorescent DNA binding dyes emit a fluorescence signal after excitation with light at a suitable wavelength. A double-stranded DNA binding dye such as a nucleic acid intercalating dye also can be used. When double-stranded DNA binding dyes are used, a melting curve analysis is usually performed for confirmation of the presence of the amplification product.

As described herein, amplification products can be detected using labeled hybridization probes that take advantage of fluorescence technology. A common format of fluorescence technology utilizes the TaqMan® technology to detect the presence or absence of an amplification product, and hence, the presence or absence of B. anthracis. TaqMan® technology utilizes one single-stranded hybridization probe labeled with two fluorescent moieties. When a first fluorescent moiety is excited with light of a suitable wavelength, the absorbed energy is transferred to a second fluorescent moiety. The second fluorescent moiety is generally a quencher molecule. During the annealing step of the PCR reaction, the labeled hybridization probe binds to the target DNA (i.e., the amplification product) and is degraded by the 5′ to 3′ exonuclease activity of the Taq Polymerase during the subsequent elongation phase. As a result, the excited fluorescent moiety and the quencher moiety become spatially separated from one another. As a consequence, upon excitation of the first fluorescent moiety in the absence of the quencher, the fluorescence emission from the first fluorescent moiety can be detected.

Molecular beacons in conjunction with fluorescence also can be used to detect the presence of an amplification product using the real-time PCR methods of the invention. Molecular beacon technology uses a hybridization probe labeled with a first fluorescent moiety and a second fluorescent moiety. The second fluorescent moiety is generally a quencher, and the fluorescent labels are typically located at each end of the probe. Molecular beacon technology uses a probe oligonucleotide having sequences that permit secondary structure formation (e.g., a hairpin). As a result of secondary structure formation within the probe, both fluorescent moieties are in spatial proximity when the probe is in solution. After hybridization to the target nucleic acids (i.e., amplification products), the secondary structure of the probe is disrupted and the fluorescent moieties become separated from one another such that after excitation with light of a suitable wavelength, the emission of the first fluorescent moiety can be detected.

It is understood that the present invention is not limited by the configuration of one or more commercially available instruments.

The invention further provides for articles of manufacture to detect B. anthracis. An article of manufacture according to the present invention can include primers and probes used to detect B. anthracis, together with suitable packaging materials. Representative primers and probes for detection of B. anthracis are capable of hybridizing to B. anthracis capA or Lef nucleic acid molecules. Methods of designing primers and probes are disclosed herein, and representative examples of primers and probes that amplify and hybridize to B. anthracis capA or Lef nucleic acid molecules are provided.

Articles of manufacture of the invention also can include one or more fluorescent moieties for labeling the probes or, alternatively, the probes supplied with the kit can be labeled. For example, an article of manufacture may include a donor fluorescent moiety for labeling one end of the capA or Lef probes and an acceptor fluorescent moiety for labeling the other end of the capA or Lef probe, respectively. Examples of suitable FRET donor fluorescent moieties and corresponding acceptor fluorescent moieties are provided above.

Articles of manufacture of the invention also can contain a package insert or package label having instructions thereon for using the capA primers and probes or Lef primers and probes to detect B. anthracis in a sample. Articles of manufacture may additionally include reagents for carrying out the methods disclosed herein (e.g., buffers, polymerase enzymes, co-factors, or agents to prevent contamination). Such reagents may be specific for one of the commercially available instruments described herein.

Thus, a first special aspect of the invention relates to a method for detecting the presence or absence of Bacillus anthracis in a biological or clinical sample from an individual or in a non-biological sample, said method comprising: performing at least one cycling step, wherein said cycling step comprises an amplifying step and a hybridizing step, wherein said amplifying step comprises contacting said sample with a pair of capA primers to produce a capA amplification product if a B. anthracis capA nucleic acid molecule is present in said sample. Mentioned capA amplification product is being amplified further by performing at least one additional cycling step, comprising contacting said sample and said capA amplification product with a pair of capA nested primers, to produce a nested capA amplification product. Mentioned hybridizing step of said method comprises contacting said sample with a capA probe, wherein the capA probe is labeled with a detectable label and detecting the presence or absence of released labeling signal, wherein the presence of released labeling signal is indicative of the presence of B. anthracis in said sample, and wherein the absence of released labeling signal is indicative of the absence of B. anthracis in said sample.

In a special embodiment of the invention, the pair of capA primers comprises a first capA primer and a second capA primer, wherein said first capA primer comprises the nucleotide sequence 5′-GGC GAA ACA TGA CGA AAA AC-3′ (SEQ ID NO:1), and wherein said second capA primer comprises the sequence 5′-CCT CGT TAT GTA GCA ATC GTA TTA C-3′ (SEQ ID NO:2) or contiguous nucleotides hereof.

In a special embodiment of the invention, the pair of nested capA primers further comprises a third capA primer and a fourth capA primer, wherein said third nested capA primer comprises the sequence 5′-TTA CGT GAC GTC CCA TC-3′ (SEQ ID NO:3), and wherein said fourth nested capA primer comprises the sequence 5′-TGC GAC ATG GGT ACA AC-3′ (SEQ ID NO:4) or contiguous nucleotides hereof.

In a special embodiment of the invention, the first capA probe comprises the sequence 5′-CAA CCA TCG TCA TCG TCA ATT-3′ (SEQ ID NO:5) or contiguous nucleotides hereof.

In a special embodiment of the invention, the detection comprises quantitation by means of said FRET or said probe-specific voltage peak (PSVP).

In a special embodiment of the invention, the detecting step is performed after each amplification or cycling step.

In a special embodiment of the invention, the detecting step is performed in real time.

In a special embodiment of the invention, the presence of said FRET detection signal within 100 cycling steps is indicative of the presence of B. anthracis.

In a special embodiment of the invention, the presence of said FRET detection signal within 50 cycling steps is indicative of the presence of B. anthracis.

In a special embodiment of the invention, the presence of said FRET detection signal within 30 cycling steps is indicative of the presence of B. anthracis.

In a special embodiment of the invention, the biological sample is derived from the group consisting of dermal swabs, nasal swabs, cerebrospinal fluid, blood, sputum, bronchio-alveolar lavage, bronchial aspirates, and feces.

In a special embodiment of the invention, the non-biological sample is selected from the group consisting of powders, filtered air samples, surface swipes, dust, dirt and soil samples and rinse products from solid materials.

In a special embodiment of the invention, the method further comprising: performing at least one cycling step, wherein a cycling step comprises an amplifying step and a hybridizing step, wherein said amplifying step comprises contacting said sample with a pair of Lef primers to produce a Lef amplification product if a B. anthracis Lef nucleic acid molecule is present in said sample. The Lef amplification product being further amplified by performing at least one additional cycling step comprising contacting said sample and said Lef amplification product with a pair of Lef nested primers to produce a nested Lef amplification product. The hybridizing step of said method comprises contacting said sample with a Lef probe, wherein the Lef probe is labeled with a detectable label and detecting the presence or absence of released labeling signal, wherein the presence of released labeling signal is indicative of the presence of B. anthracis in said sample, and wherein the absence of released labeling signal is indicative of the absence of B. anthracis in said sample.

In a special embodiment of the invention, the pair of Lef primers comprises a first Lef primer and a second Lef primer, wherein said first Lef primer comprises the sequence 5′-AAA AGG TAA CAA ATT ACT TAG TTG ATG G-3′ (SEQ ID NO:6), and wherein said second Lef primer comprises the sequence 5′-CGA AGT TAA ATT ACT CCC TTC TTC CTT-3′ (SEQ ID NO:7) or contiguous nucleotides hereof.

In a special embodiment of the invention, the pair of nested Lef primers comprises a third Lef primer and a fourth Lef primer, wherein said third Lef primer comprises the sequence 5′-GGG TTA TAT GTT CCA GAA TC-3′ (SEQ ID NO:8), and wherein said fourth Lef primer comprises the sequence 5′-GTA ACT AAA TCA GAT TGG TTC T-3′ (SEQ ID NO:9) or contiguous nucleotides hereof.

In a special embodiment of the invention, the first Lef probe comprises the sequence 5′-GAC CTT CAA AAG GTG TAG AAT TAA GG-3′ (SEQ ID NO:10) or contiguous nucleotides hereof.

In a special embodiment of the invention, the cycling step is performed on a control sample.

In a special embodiment of the invention, the control sample comprises said portion of said B. anthracis capA nucleic acid molecule.

In a special embodiment of the invention, the cycling step uses a pair of control primers and a control probe, wherein said control primers and said control probe are other than said capA primers and capA probe, wherein said amplifying step produces a control amplification product, wherein said control probes hybridize to said control amplification product.

A further special aspect of the invention relates to a method for detecting the presence or absence of B. anthracis in a biological sample from an individual or in a non-biological sample, said method comprising: performing at least one cycling step, wherein a cycling step comprises an amplifying step and a hybridizing step, wherein said amplifying step comprises contacting said sample with a pair of capA primers to produce a capA amplification product if a B. anthracis capA nucleic acid molecule is present in said sample. The capA amplification product being further amplified by performing at least one additional cycling step comprising contacting said sample and said capA amplification product with a pair of capA nested primers to produce a nested capA amplification product. The hybridizing step of said method comprises contacting said sample with a capA probe, wherein the capA probe is labeled with a donor fluorescent moiety and a corresponding acceptor fluorescent moiety; and detecting the presence or absence of Foster Resonance Energy Transfer (FRET) between said donor fluorescent moiety and said acceptor fluorescent moiety of said capA probe, wherein the presence of FRET is indicative of the presence of B. anthracis in said sample, and wherein the absence of FRET is indicative of the absence of B. anthracis in said sample.

Yet a further special aspect of the invention relates to a method for detecting the presence or absence of B. anthracis in a biological sample from an individual or in a non-biological sample, said method comprising: performing at least one cycling step, wherein a cycling step comprises an amplifying step and a hybridizing step, wherein said amplifying step comprises contacting said sample with a pair of Lef primers to produce a Lef amplification product if a B. anthracis Lef nucleic acid molecule is present in said sample. The Lef amplification product being further amplified by performing at least one additional cycling step comprising contacting said sample and said Lef amplification product with a pair of Lef nested primers to produce a nested Lef amplification product. The hybridizing step of said method comprises contacting said sample with a Lef probe, wherein the Lef probe is labeled with a donor fluorescent moiety and a corresponding acceptor fluorescent moiety; and detecting the presence or absence of Foster Resonance Energy Transfer (FRET) between said donor fluorescent moiety and said acceptor fluorescent moiety of said Lef probe, wherein the presence of FRET is indicative of the presence of B. anthracis in said sample, and wherein the absence of FRET is indicative of the absence of B. anthracis in said sample.

A further special aspect of the present invention relates to article of manufacture, comprising: a pair of capA primers; a pair of nested capA primers; a capA probe; and a donor fluorescent moiety and a corresponding acceptor fluorescent moiety.

In a special embodiment of the invention, the pair of capA primers comprises a first capA primer and a second capA primer, wherein said first capA primer comprises the sequence 5′-GGC GAA ACA TGA CGA AAA AC-3′ (SEQ ID NO:1), and wherein said second capA primer comprises the sequence 5′-CCT CGT TAT GTA GCA ATC GTA TTA C-3′ (SEQ ID NO:2) or contiguous nucleotides hereof.

In a special embodiment of the invention, the pair of nested capA primers further comprises a third capA primer and a fourth capA primer, wherein said third nested capA primer comprises the sequence 5′-TTA CGT GAC GTC CCA TC-3′ (SEQ ID NO:3), and wherein said fourth nested capA primer comprises the sequence 5′-TGC GAC ATG GGT ACA AC-3′ (SEQ ID NO:4) or contiguous nucleotides hereof.

In a special embodiment of the invention, the first capA probe comprises the sequence 5′-CAA CCA TCG TCA TCG TCA ATT-3′ (SEQ ID NO:5) or contiguous nucleotides hereof.

In a special embodiment of the invention, the capA probe is labeled with said donor fluorescent moiety and said corresponding acceptor fluorescent moiety.

In a special embodiment of the invention, the article of manufacture further comprises a package insert having instructions thereon for using said pair of capA primers, said pair of nested capA primers, and said capA probe to detect the presence or absence of B. anthracis in a sample.

Yet a further special aspect of the invention relates to an article of manufacture, comprising: a pair of Lef primers; a pair of nested Lef primers; a Lef probe; and a donor fluorescent moiety and a corresponding acceptor fluorescent moiety.

In a special embodiment of the invention, the pair of Lef primers comprises a first Lef primer and a second Lef primer, wherein said first Lef primer comprises the sequence 5′-AAA AGG TAA CAA ATT ACT TAG TTG ATG G-3′ (SEQ ID NO:6), and wherein said second Lef primer comprises the sequence 5′-CGA AGT TAA ATT ACT CCC TTC TTC CTT-3′ (SEQ ID NO:7) or contiguous nucleotides hereof.

In a special embodiment of the invention, the pair of nested Lef primers comprises a third Lef primer and a fourth Lef primer, wherein said third Lef primer comprises the sequence 5′-GGG TTA TAT GTT CCA GAA TC-3′ (SEQ ID NO:8), and wherein said fourth Lef primer comprises the sequence 5′-GTA ACT AAA TCA GAT TGG TTC T-3′ (SEQ ID NO:9) or contiguous nucleotides hereof.

In a special embodiment of the invention, the first Lef probe comprises the sequence 5′-GAC CTT CAA AAG GTG TAG AAT TAA GG-3′ (SEQ ID NO:10) or contiguous nucleotides hereof.

Another special aspect of the invention relates to a method for detecting the presence or absence of B. anthracis in a biological sample from an individual or in a non-biological sample, said method comprising: performing at least one cycling step, wherein a cycling step comprises an amplifying step and a hybridizing step, and a dye-binding step wherein said amplifying step comprises contacting said sample with a pair of capA primers to produce a capA amplification product if a B. anthracis capA nucleic acid molecule is present in said sample. The capA amplification product being further amplified by performing at least one additional cycling step comprising contacting said sample and said capA amplification product with a pair of capA nested primers to produce a nested capA amplification product, wherein said dye-binding step comprises contacting said capA amplification product with a double-stranded DNA binding dye; and detecting the presence or absence of binding of said double-stranded DNA binding dye into said amplification product, wherein the presence of binding is indicative of the presence of B. anthracis in said sample, and wherein the absence of binding is indicative of the absence of B. anthracis in said sample.

Still another special aspect of the invention relates to a method for detecting the presence or absence of B. anthracis in a biological sample from an individual or in a non-biological sample, said method comprising: performing at least one cycling step, wherein a cycling step comprises an amplifying step and a hybridizing step, and a dye-binding step wherein said amplifying step comprises contacting said sample with a pair of Lef primers to produce a Lef amplification product if a B. anthracis Lef nucleic acid molecule is present in said sample. The Lef amplification product being further amplified by performing at least one additional cycling step comprising contacting said sample and said Lef amplification product with a pair of Lef nested primers to produce a nested Lef amplification product, wherein said dye-binding step comprises contacting said Lef amplification product with a double-stranded DNA binding dye; and detecting the presence or absence of binding of said double-stranded DNA binding dye into said amplification product, wherein the presence of binding is indicative of the presence of B. anthracis in said sample, and wherein the absence of binding is indicative of the absence of B. anthracis in said sample.

In a special embodiment of the invention, the double-stranded DNA binding dye is selected from the group consisting of SYBRGreen I®, SYBRGold®, and ethidium bromide.

In a special embodiment of the invention, the method, further comprising determining the melting temperature between said capA amplification product and said double-stranded DNA binding dye, wherein said melting temperature confirms said presence or absence of said B. anthracis.

It should be noted that, according to the present invention, embodiments and features described in the context of one of the aspects of the present invention also apply to the other aspects of the invention.

### EXAMPLES

### Example 1

### Oligonucleotide Primers and Probes

Primers and probes were designed using the software VectorNTI™ version 5.0 (Informax, Inc., Frederick, Md.). Sequences for primers and probes are shown in Table 3. The GenBank Accession numbers for the reference sequences used to design the primers and probes for each target are shown in Table 3, along with the relative location of each primer and probe.

TABLE 3

| | | | | | |
|---|---|---|---|---|---|
| | Seq | | | Length | Tₘ |
| Primer | ID | Name | Sequence | bases | ° C. |

| | | | | | |
|---|---|---|---|---|---|
| pXO2 outer sense | 1 | PcapAlnS | 5′-GGCGAAACATGACGAAAAAC-3′ | 20 | 60.1 |
| primer | | | | | |
| pXO2 outer antisense | 2 | PcapAlnA | 5′-CCTCGTTATGTAGCAATCGTATTAC-3′ | 25 | 59.4 |
| primer | | | | | |
| pXO2 inner sense | 3 | PcapAnesS | 5′-TTACGTGACGTCCCATC-3′ | 17 | 51.2 |
| primer | | | | | |
| pXO2 inner antisense | 4 | PcapAnesA | 5′-TGCGACATGGGTACAAC-3′ | 17 | 52.2 |
| primer | | | | | |
| PROBE-pXO2 | 5 | PcapAprobe | 5′-FAM-CAACCATCGTCATCGTCAATT-BHQ-3′ | 21 | 60.2 |
| pXO1 outer sense | 6 | PXouts | 5′-AAAAGGTAACAAATTACTTAGTTGATGG-3′ | 28 | 60.5 |
| primer | | | | | |
| pXO1 outer antisense | 7 | PXouta | 5′-CGAAGTTAAATTACTCCCTTCTTCCTT-3′ | 27 | 63.4 |
| primer | | | | | |
| pXO1 inner sense | 8 | PXIns | 5′-GGGTTATATGTTCCAGAATC-3′ | 20 | 50.6 |
| primer | | | | | |
| pXO1 inner antisense | 9 | PXIna | 5′-GTAACTAAATCAGATTGGTTCT-3′ | 22 | 49.9 |
| primer | | | | | |
| PROBE-pXO1 | 10 | PX-probe | 5′-FAM-GACCTTCAAAAGGTGTAGAATTAAGG- | 26 | 61.2 |
| | | | BHQ-3′ | | |
| pXO1 primer gives 912 | 11 | PXS | 5′-AATATCAATAACCTTACAGCAACCC-3′ | 25 | 60.0 |
| bp product with PXA | | | | | |
| pXO1 primer gives 912 | 12 | PXA | 5′-ATGCATTAACCTAAAGGCTTCTG-3′ | 23 | 59.6 |
| bp product with PXS | | | | | |
| Alternative probe for | 13 | | 5′-CCA TCG TCA TCG TCA AT-3′ | 17 | |
| capA on pXO2 | | | | | |
| Alternative probe for | 14 | | 5′-TCA AAA GGT GTA GAA TTA AGG-3′ | 21 | |
| Lef on pXO1 | | | | | |

FAM is a fluorescent dye (a carboxyfluorescein with an absorption peak at 492 nm and an emission peak at 515 nm) and BHQ™ is a quencher (Black Hole Quencher). FAM and BHQ are used for tests using on the real-time PCR machine.

The pXO2 initial sense and antisense primer set amplifies a 209 base pair region. The pXO1 outer sense and antisense primer set amplifies a 335 base pair region. Primers were adjusted to a stock solution of 100 μM.

Probes were dissolved in TE-buffer to a concentration of 20 μM (supplied with the probes and resuspended according to manufacturer's instructions).

The analytical sensitivities for the two gene targets (i.e., capA or lef) were at least 10 copies of the target sequence.

### Example 2

PCR Conditions

The DNA Engine Opticon™ hybridization mixture was identical for each B. anthracis gene target (with the exception that each primer and probe set was specific for the particular gene target that was amplified).

A standard 1×DNA Engine Opticon™ hybridization mixture for B. anthracis capA or Lef comprises the following (Karsai et al.):

10 mM TrisHCl, pH 8.5

50 mM KCl

2 mM MgCl₂

0.1% Tween-20 and/or 0.15% Triton X-100

±20 μg/ml of BSA

±0.3% DMSO or 0.8% glycerol

The DNA Engine Opticon™ thermocycling conditions were identical for each gene target and are listed in Table 4.

TABLE 4

| Number of cycles | Comment | Temperature (° C.) | time |
|---|---|---|---|
| 1 | Denaturation | 95.0 | 5 min |
| 50 | Outer primers | 95.0 | 10 sec |
| | | 63.0 | 30 sec |
| 51 | Inner primers | 95.0 | 10 sec |
| | | 51.0 | 30 sec |
| | | 60.0 | 30 sec |
| | | 4 | Until stopped |

To generate the 912 bp pXO1 template, DNA was isolated from an overnight culture of Bacillus anthracis grown on solid substrate. Colonies was scraped of, resuspended in TE-buffer and boiled for 15 minutes in sealed PCR tubes using a heated lid thermocycler. Subsequently, the boiled culture was phenol extracted and DNA was precipitated and washed with 70% ethanol prior to dissolving in TE-buffer. The purified DNA was used for a PCR reaction involving the pXO1 outer sense and antisense primer set.

The concentration of the template was determined by spectrophotometric fluorescence compared to a standard. The concentration (in μg/ml) was the calculated to number of copies using

$${N}_{A} = 6.023 {10}^{23} \frac{( \% GC * total length )}{100} ( 618.4 ) + \frac{( 100 - \% GC * total length )}{100} ( 617.4 ) + 36.0$$

The 912 bp template generated using primers PXA and PXS comprises a G+C content of 30.15% and subsequently an A+T content of 69.85%. Using the above equations, this gives a molecular weight of 563,379 Daltons, i.e., one single molecule weighs 9.4 10⁻¹⁹ g≈1 ag. The purified 912 bp template stock had a concentration of 1 μg/ml giving the following concentrations and number of molecules in the working solutions:

| | | | Number of molecules | Number of molecules |
|---|---|---|---|---|
| Dilution | Concentration | | per μl | per reaction* |
| 10⁻³ | 1 | pg/μl | 10⁶ | 1.67 10⁵ |
| 10⁻⁴ | 0.1 | pg/μl | 10⁵ | 1.67 10⁴ |
| 10⁻⁵ | 0.01 | pg/μl | 10⁴ | 1.67 10³ |
| 10⁻⁶ | 1 | fg/μl | 10³ | 167 |
| 10⁻⁷ | 0.1 | pg/μl | 10² | 16.7 |
| 10⁻⁸ | 0.01 | pg/μl | 10 | 1.67 |

*Setting up the PCR was done by adding 6 μl of diluted template in 714 μl PCR reaction mixture and running in reaction volumes of 20 μl (i.e., a dilution factor of 120/20 = 6)

Using a working solution of template diluted 10⁻⁷ (i.e., approximately 17 molecules per reaction), the definite identification of the small number of template molecules was detected in the single tube nested PCR reaction. Furthermore, as evident from the figure, the single tube nested PCR reaction was able to detect the template 3 cycles before the “non-nested” (i.e., no outer primers) reaction—thus exhibiting a 10-fold increase in sensitivity.

### Example 3

Melting Curves

Following the completion of the amplification reactions, a melting curve analysis can be performed. The determination of Tm can be used to check the specificity of an amplified product. Using SYBR® Green fluorescence during a temperature increase, a decrease in fluorescence is observed as the product undergoes denaturation. The temperature in the DNA Engine Opticon™ thermal chamber is raised from 50° C. to 85° C. at 0.2° C. per second. Fluorescent measurements is taken continuously as the temperature is raised and melting curves is generated. Each product has a specific and characteristic melting curve from the respective PCR reactions. Optionally, the melting curve determination can be followed by re-annealing at 72° C. for 5-10 min if the samples are to be analyzed e.g. using agarose gel electrophoresis.

### REFERENCES

Beyer et al. Beyer W, Glockner P, Otto J, Bohm R. (1995) A nested PCR method for the detection of Bacillus anthracis in environmental samples collected from former tannery sites. Microbiol Res. 150:179-86.
Herrmann et al. Herrmann B, Nystrom T, Wessel H. (1996) Detection of Neisseria gonorrhoeae from air-dried genital samples by single-tube nested PCR. J Clin Microbiol. 34:2548-51.
Jackson et al. Jackson P I, Hugh-Jones M E, Adair D M, Green G, Hill K K, Kuske C R, Grinberg L M, Abramova F A, Keim P. (1998) PCR analysis of tissue samples from the 1979 Sverdlovsk anthrax victims: the presence of multiple Bacillus anthracis strains in different victims. Proc Natl Acad Sci USA. 95:1224-9.
Karsai et al. Karsai A, Muller S, Platz S, Hauser M T. (2002) Evaluation of a homemade SYBR green I reaction mixture for real-time PCR quantification of gene expression. Biotechniques. 32:790-2, 794-6.
Rasmussen et al. Rasmussen, R. P., and Wittwer, C. T. (1997) Product Differentiation by Analysis of DNA Melting Curves during the Polymerase Chain Reaction. Analytical Biochemistry, 245:154-160
Sambrook et al: Molecular cloning: a Laboratory Manual: 3nd edition, Volume 1 and 2, Sambrook et al., 2001, Cold Spring Harbor Laboratory Press

1. A method of amplifying, and optionally also detecting, a target nucleic acid sequence, the method comprising the steps of:
a) providing a sample that may or may not comprise a target nucleic acid sequence,
b) providing a pair of outer primers and a pair of inner primer, a nucleic acid polymerase and standard reagents for PCR, the melting temperature (Tm) of the pair of outer primers being at least 2° C. higher than the Tm of the pair of inner primers,
c) contacting the sample with the pair of outer primer and the pair of inner primers, and standard reagents for PCR, thus obtaining the reaction mixture,
d) cycling, at least two times, the temperature of the reaction mixture between a first denaturation temperature, a first annealing temperature and a first extension temperature, the first annealing temperature being approximately the same as or lower than the lowest Tm of the outer primer pair and higher than the highest Tm of the inner primer pair, and
e) cycling, at least two times, the temperature of the reaction mixture between a second denaturation temperature, a second annealing temperature and a second extension temperature, the second annealing temperature being similar to or lower than the lowest Tm of the inner primer pair.

2. The method according to claim 1, wherein the Tm of the pair of outer primers is 2-10° C. higher than the Tm of the pair of inner primers.

3. The method according to claim 1, wherein at least one primer of the outer primer pair comprises a Tm-increasing component.

4. The method according to claim 1, wherein both of the primers of the outer primer pair comprise a Tm-increasing component.

5. The method according to claim 4, wherein the Tm-increasing component binds non-specifically to nucleic acids.

6. The method according to claim 3, wherein the Tm-increasing component comprises one or more moieties selected from the group consisting of a modified nucleotide and a minor groove binding agent.

7. The method according to claim 6, wherein the modified nucleotide is a peptide nucleic acid (PNA) or a locked nucleic acid (LNA).

8. The method according to claim 3, wherein the Tm-increasing component increases the Tm of the primer with at least 1° C. relative to the Tm of the same primer not comprising the Tm-increasing component.

9. The method according to claim 1, wherein the second denaturation temperature is at least 1° C. lower than the first denaturation temperature.

10. A method for detection of Bacillus anthracis, the method comprising detecting a target nucleic acid sequence according to the method of claim 1, the target nucleic acid sequence being specific for the pXO1 or pXO2 plasmid of Bacillus anthracis, wherein the pair of outer primers and the pair of inner primers are selected from the pXO1 or pXO2 plasmid of Bacillus anthracis.

11. The method according to claim 10, wherein the pair of outer primers and the pair of inner primers are selected to amplify a target nucleic acid sequence related to a gene selected from the group of B. anthracis genes consisting of capA gene, the capB gene, the capC gene, the lef gene.

12. The method according to claim 10, wherein target nucleic acid sequence is related to the capA gene and
a primer of the pair of outer primers comprises a nucleic acid sequence selected from the group of SEQ ID NO: 1, SEQ ID NO: 2, a homologous sequence thereof, and a complementary sequence thereof, and
a primer of the pair of inner primers comprises a nucleic acid sequence selected from the group of SEQ ID NO: 3, SEQ ID NO: 4, a homologous sequence thereof, and a complementary sequence thereof.

13. The method according to claim 10, wherein target nucleic acid sequence is related to the capA gene and the pair of outer primers comprises SEQ ID NOs: 1 and 2 and/or the pair of inner primers comprises SEQ ID NOs: 3 and 4.

14. A kit comprising a pair of outer primers and a pair of inner primer, the melting temperature (Tm) of the pair of outer primers being higher than the Tm of the pair of inner primers.

15. The kit according to claim 14, wherein the Tm of the pair of outer primers is 2-10° C. higher than the Tm of the pair of inner primers.

16. The kit according to claim 14, wherein at least one primer of the outer primer pair comprises a Tm-increasing component.

17. The kit according to claim 16, wherein both of the primers of the outer primer pair comprises a Tm-increasing component.

18. The kit according to claim 16, wherein the Tm-increasing component binds non-specifically to nucleic acids.

19. The kit according to claim 16, wherein the Tm-increasing component comprises one or more moieties selected from the group consisting of a modified nucleotide and a minor groove binding protein.

20. The kit according to claim 19, wherein the modified nucleotide is a peptide nucleic acid (PNA) or a locked nucleic acid (LNA).

21. The kit according to claim 16, wherein the Tm-increasing component increases the Tm of the primer with at least 1° C. relative to the Tm of the same primer not comprising the Tm-increasing component.

22. A kit according to claim 14, for detection of Bacillus anthracis, the kit comprising a pair of outer primers and a pair of inner primer, the melting temperature (Tm) of the pair of outer primers being higher than the Tm of the pair of inner primers, wherein the pair of outer primers and the pair of inner primers are selected from the pXO1 or pXO2 plasmid of Bacillus anthracis.

23. The kit of claim 22, wherein the pair of outer primers and the pair of inner primers are selected so as to amplify a target nucleic acid sequence within a gene selected from the group of B. anthracis genes consisting of capA gene, the Cap B gene, the Cap C gene, the lef gene.

24. The kit according to claim 22, wherein target nucleic acid sequence is related to the capA gene and
a primer of the pair of outer primers comprises a nucleic acid sequence selected from the group of SEQ ID NO: 1, SEQ ID NO: 2, a homologous sequence thereof, and a complementary sequence thereof, and
a primer of the pair of inner primers comprises a nucleic acid sequence selected from the group of SEQ ID NO: 3, SEQ ID NO: 4, a homologous sequence thereof, and a complementary sequence thereof.

25. The kit according to claim 22, wherein target nucleic acid sequence is related to the capA gene and the pair of outer primers comprises SEQ ID NOs: 1 and 2 and/or the pair of inner primers comprises SEQ ID NOs: 3 and 4.

26. An analysis system for detection of a microorganism, the analysis system comprising a pair of outer primers and a pair of inner primer, the melting temperature (Tm) of the pair of outer primers being higher than the Tm of the pair of inner primers.

27. The analysis according to claim 26, wherein the Tm of the pair of outer primers is 2-10° C. higher than the Tm of the pair of inner primers.

28. The analysis system according to claim 26, wherein at least one primer of the outer primer pair comprises a Tm-increasing component.

29. The analysis system of claim 26, wherein the analysis system is selected from the group consisting of a lateral flow device, a biochip, and a microarray.

30. The method of claim 1, further comprising analyzing the product of step d) and/or step e) to detect the presence of the target nucleic acid sequence.
